(19) United States
(12) Patent Application Publication
(10) Pub. No.: US 2025/0152476 A1
(43) Pub. Date: May 15, 2025

(54) COMPANION INTERACTIVE INTELLIGENT HEALTH CARE SYSTEM AND INTELLIGENT MEDICATION MANAGEMENT METHOD THEREOF

(71) Applicant: MJOLNIR ROBOTS LIMITED, Grand Cayman (KY)

(72) Inventor: YI SHEN, Toronto (CA)

(21) Appl. No.: 18/940,930

(22) Filed: Nov. 8, 2024

Related U.S. Application Data
(60) Provisional application No. 63/598,170, filed on Nov. 13, 2023.

(51) Int. Cl. A61J 7/04; G16H 20/13

(52) U.S. Cl. CPC A61J 7/04; G16H 20/13

(57) ABSTRACT

A companion interactive intelligent health care system is provided. The system includes a multifunctional intelligent medicine box, a companion interactive intelligent robot, and an intelligent display processing terminal. The multifunctional intelligent medicine box is used to display the name and dosage information of medicine, indicate the location of the medicine, and record the medication information. The companion interactive intelligent robot integrates voice, expression, and action, and is used to remind the user to take medicine on time and in dosage through multiple sensory interactions, provide emotional companionship, and remote calls with family members. The intelligent display processing terminal is used for system software operation, data acquisition, storage, and analysis, remote viewing, and monitoring of user medication records. The present invention also provides an intelligent medication management method based on the above-mentioned companion interactive intelligent health care system, which has a wide range of application scenarios.

### CROSS-REFERENCE TO RELATED APPLICATION

This non-provisional patent application claims the benefit of U.S. provisional patent application No. 63/598,170, filed on Nov. 13, 2023, the content of which is incorporated herein by reference in their entirety.

### BACKGROUND

### Technical Field

The present disclosure relates to a companion interactive smart health care system and an intelligent medication management method thereof.

### Related Art

With the advent of an aging society, how to give the elderly a happy old age is a common concern of the government and society. One of the key points and difficulties of healthy home-based elderly care is to take medicine on time and in the right amount. Taking medicine on time and in the right amount is the primary issue of healthy elderly care. Traditional medicine boxes only have the function of storing medicines, lack intelligent medicine management functions, and cannot guide the elderly to take the correct medicines on time. The elderly often suffer from various health problems due to wrong or missed medications. At the same time, mastering the daily health status data of the elderly is one of the important measures to ensure healthy elderly care. Many families have configured daily health status detection equipment such as blood sugar, blood pressure, and blood oxygen, but since most of the equipment comes from different manufacturers, the scattered APP calls have caused great inconvenience to the elderly and their relatives. In addition, with the progress of society, the emotional attention to the elderly is constantly increasing, and there is a need for smart products that can realize vivid communication and interaction with the elderly, accompany them, and relieve the emotional loneliness and anxiety of the elderly. Existing smart interactive products are mostly based on voice query functions, with simple expressions and actions or no expressions and actions. Based on the above-mentioned interaction needs, developing humanoid robots, improving the flexibility of robot structures, and enhancing the friendliness of human-machine interaction are the problems and directions that urgently need to be solved and explored in current robot structure design.

Therefore, a large number of smart medication management products have emerged on the market in recent years, but the existing products lack multifunctional smart medication management products that have voice reminders for medication, screen displays of medication information, and connections to mobile applications.

### SUMMARY

In order to solve the deficiencies in the prior art, the purpose of the present disclosure is to provide a companion interactive intelligent health care system and an intelligent medication management method thereof. Addressing issues such as health monitoring and management, home environment monitoring and early warning, as well as the lack of interactive companionship, we aim to bridge the gap in integrated technology solutions. For instance, we explore how smart devices can be utilized to prevent excessive or insufficient medication in the elderly, thereby controlling the progression and deterioration of chronic diseases and minimizing the occurrence of complications. Additionally, we investigate how smart devices can be integrated into home environments to facilitate home-based exercise rehabilitation training, adhering to scientific and moderate exercise regimens to enhance the quality of life. Furthermore, we explore how various age-friendly products can be seamlessly integrated with the system to promptly detect and treat diseases, addressing other health-related issues among the elderly.

The companion interactive intelligent health care system proposed by the present disclosure applies independently designed hardware applied to smart health care usage scenarios, and combines technologies such as the Internet of Things and artificial intelligence to provide a comprehensive implementation solution in the field of smart health care, including health monitoring and management, medication management, interactive companionship, environmental monitoring and early warning.

Further, the companion interactive intelligent health care system includes a hardware part and a software part.

The hardware part includes a multifunctional intelligent medicine box, a companion interactive intelligent robot, and an intelligent display processing terminal.

The multifunctional intelligent medicine box is used to display the name and dosage information of the medicine, indicate the medicine compartment corresponding to the medicine, and record the medication information.

The companion interactive intelligent robot integrates voice, expression, and action, and is used to remind a user to take medicine on time and in the right amount through multiple sensory interactions, and provides emotional companionship and remote communication with family members.

The intelligent display processing terminal is used for system software operation, data acquisition and/or storage and/or analysis, and remote viewing and/or monitoring of user people's medication records.

The software part integrates Internet of Things technology and artificial intelligence technology and comprises a health care cloud platform and/or a family end and/or a service back end and/or a home intelligent end and/or an intelligent medicine box end and/or a robot end.

The multifunctional intelligent medicine box proposed by the present disclosure is an intelligent medication management product that combines voice, display, WIFI access to cloud platforms, expandable structure, and locatable medicine storage locations. It meets the needs of hearing, vision, remote monitoring, storage, and management of medication, and is suitable for different scenarios and populations.

The present disclosure provides a multifunctional intelligent medicine box with an expanded structure. The multifunctional intelligent medicine box includes a machine head and at least one medicine storage box.

The machine head is a medicine box display and control terminal, including a machine head body and a machine head base; the machine head body and the machine head base are integrated structures.

The medicine storage box includes a box shell and a medicine storage drawer. The medicine storage drawer can be pulled out and moved inside the box shell.

The machine head matches the top structure of the medicine storage box, so that the machine head is fixedly connected to the medicine storage box.

The front of the head body is provided with a display screen, a scanning camera, function buttons, and a volume adjustment button;

In a specific embodiment, the display screen can display medicine name, dosage, current time, battery level, charging mode, WIFI connection status, Bluetooth connection status, or any combination thereof;
the scanning camera is arranged above the display screen, and can scan the barcode of the medicine package to obtain medicine information for medication management.
the volume adjustment button is arranged on the right side of the display screen and is used to adjust the voice broadcast volume;
the function button is set below the display screen, which can turn on/off the voice broadcast; long press to activate the camera scanning function, scan the barcode on the medicine package, and obtain medicine information.

A control mainboard and the power control board are fixedly arranged at the back of the display screen inside the machine head body; the control mainboard comprises a voice module, a display module, and a WIFI module;
the voice module is used to recognize and generate sounds; the display module is used to display the status information of the medicine box device, medication-related information and input medication information; the WIFI module is used to transmit device information and medication management information to the cloud platform;
the power control board can serve as a transfer structure for electrical connection and can monitor battery power, power connection status, and machine switch status;
a lithium battery, a Bluetooth antenna array board, and a speaker cavity are fixedly arranged at the bottom of the interior of the machine head body; the speaker cavity is installed above the Bluetooth antenna array board and corresponds to the position of the speaker hole on the back of the machine head body;

The LED light status signal and the button status signal are sent to the Bluetooth control board through wires, and the Bluetooth control board sends the medicine compartment position signal to the Bluetooth antenna array board of the machine head through Bluetooth, and the Bluetooth antenna array board sends the signal to the main control board through wires, and the main control board sends the position signal to the cloud platform through WIFI.

The control mainboard, the power control board, the lithium battery, the Bluetooth antenna array board, and the speaker cavity are electrically connected, and the processed signals transmitted therebetween are aggregated to the control main board;
the control main board may also be optionally provided with a Bluetooth module as a backup structure, which is used to transmit information between the machine head and the medicine storage box through Bluetooth; on one hand, the Bluetooth module can be enabled when the Bluetooth antenna array board fails due to various reasons; on the other hand, the Bluetooth module can be used in subsequent multi-channel signal upgrades;

The lower side of the back of the machine head body is also provided with a power switch, a power interface, and a network distribution button;
the power switch is electrically connected to the power control board, and can be used to control the start and shut down of the machine head and/or the medicine storage box by turning the power on and off;
the power interface is electrically connected to the power control board and is used to connect external wires to directly power the machine head and/or charge the lithium battery in the machine head;
the network distribution button is electrically connected to the control main board and is used to connect to the WIFI.

In a specific implementation, the machine head can also adopt the following structure: a display screen receiving cassette is set at the upper end of the machine head base, and the display screen matches the size of the display screen receiving cassette; the display screen is connected to the machine head base through hinges and damping support, and can be stored in the display screen receiving cassette; the damping support is installed on the back of the display screen to control the lifting angle of the display screen, with a lifting angle range of 0 to 90 degrees.

The rear portion of the box shell is provided with a box bottom pin, a power distribution board, and a Hall induction magnet.

The medicine storage drawer comprises: a medicine storage drawer body; the medicine storage drawer body comprises: a front panel and a drawer body; the front panel is installed at the front end of the drawer body;
the front panel is provided with a drawer handle, and the side surface of the drawer body is provided with a sliding groove; the sliding groove corresponds to a box slide rail provided inside the box shell;
a medicine box status LED light is provided at the top of the front panel, and the medicine box status LED light can display different colors according to different scenes.

Inside the medicine storage drawer body is divided into medicine compartments of different sizes by pluggable partition boards, which can be used to store packaged medicines of different sizes, and the size of the medicine compartment can be customized according to actual needs; a protrusion is provided on the upper part of the partition board of each medicine compartment, and a partition board LED button light is provided at the position of the protrusion; after putting in and taking out medicines, the partition board LED button light can be manually pressed, the partition board LED button light lights up, and the position signal is sent to the control main board in the machine head via Bluetooth, the display screen displays the medicine compartment position, and the medicine compartment position is sent to the cloud platform at the same time, and is synchronously displayed on the mobile terminal APP; a partition board LED button light backboard is provided at a position on the back of the partition board corresponding to the partition board LED button light for controlling the partition board LED button light. The present disclosure can also use a high-sensitivity pressure sensor to automatically identify taking and putting medicines.

The medicine storage drawer further comprises: a Hall sensor board and a Bluetooth main control board; the Hall sensor board is installed at the rear of the medicine storage drawer body, and the Bluetooth main control board is installed at the bottom of the medicine storage drawer body;
the Hall sensor board is electrically connected to the Bluetooth main control board at the bottom of the medicine storage drawer; the Hall sensor board corresponds to the position of the Hall sensor magnet provided on the box shell, and senses the opening and closing state of the medicine storage drawer through the contact between the Hall sensor board and the Hall sensor magnet, and transmits the state signal to the Bluetooth main control board through the wire;
the Bluetooth main control board is also electrically connected to the back panel of the partition board LED button light, used to supply power to the partition board LED button light. The LED light status signal and button status signal are sent to the Bluetooth control board through wires, the Bluetooth control board sends the medicine grid position signal to the head Bluetooth antenna array board through Bluetooth, and the Bluetooth antenna array board sends the signal to the main control board through wires. The main control board sends the position signal to the cloud through WIFI.

The Bluetooth main control board receives the status signal of the partition board LED button light and the opening and closing status signal of the medicine box by the Hall sensor, and sends the signal to the Bluetooth antenna array board and/or the Bluetooth module of the machine head, and then the Bluetooth antenna array board sends the signal to the control main board of the machine head; the Bluetooth main control board supplies power to the partition board LED button light and the Hall sensor board.

The scalable structure of the spring power line ensures that power can still be supplied when pushing or pulling the drawer.

When there is more than one medicine storage box in the multifunctional intelligent medicine box, different medicine storage boxes are connected through a T-shaped structural base set at the bottom of the outer shell of the medicine storage box. The box mounting buckle set at the bottom of the T-shaped structural base is matched with the U-shaped slide rail structure and mounting slot on the top of the adjacent medicine storage box to connect and fix; the Bluetooth control panels of different medicine storage drawers can communicate with the Bluetooth antenna array on the head to realize the positioning and automatic numbering of multiple medicine storage boxes, and multiple medicine storage boxes can also be manually numbered.

The multifunctional intelligent medicine box of the present disclosure can store medicines whether packaged or not because the space divided in the medicine storage drawer is relatively large.

In a specific implementation, a medicine box attitude testing limit switch is also provided inside the medicine box, and the medicine box attitude testing limit switch can be used to count the number of opening and closing times of the medicine box.

The outer surface of the intelligent medicine box can also be equipped with a logo as needed, which can emit light.

The surface and internal structures of the intelligent medicine box can be adjusted according to actual usage needs and scenarios to meet corresponding usage requirements; in addition, some structures can also be added or deleted according to actual needs.

The companion interactive intelligent robot in the present disclosure comprising: a head mechanism, a neck mechanism, and a body mechanism; in implementation, if more precise movements are required, structures such as limbs can also be added.

The head mechanism for an intelligent robot comprises: a head bracket, a head body, a head shell, a display screen and a head bottom shell; the head body, the head shell, the display screen and the head bottom shell are all fixed on the head bracket; wherein,
the head shell includes a mask and a head back shell;
the head body includes a main control board, a capacitive touch sensor, a head DC motor and its transmission structure; the capacitive touch sensor is fixed in the head shell;
the head DC motor and its transmission structure comprises: a head DC motor, a head motor frame, a head gear set and a bearing; the head DC motor drives the head gear set to rotate, and the head gear set is connected to the bearing; the bearing is arranged on the head bottom shell; the head DC motor controls the shaking motion of the head mechanism, and is fixed in the head motor frame; the head motor frame is fixed on the head bottom shell;
the capacitive touch sensor communicates with the main control board to transmit touch-sensing signals, and the main control board controls the movement of the head DC motor;
the display screen is arranged inside the head body, can be made of materials such as LCD, OLED, etc., and the main control board is fixed at the back thereof.

In the present disclosure, the head mechanism further includes a microphone and a camera; the microphone and the camera are arranged on the head bracket, and are both communicatively connected with the main control board.

In the present disclosure, a microphone hole is provided on the mask.

The camera can be used to achieve vision-based robot positioning and rotation functions, ensuring that the robot always faces the user; furthermore, the camera can be equipped with a 200W infrared night vision camera, which is convenient for use in low light conditions at night.

In the present disclosure, the maximum rotation angle of the head mechanism to the left and right sides is 180 degrees.

In the present disclosure, a capacitive touch sensor senses the user's touch on the head shell, and one is set on each side and top of the head shell.

In the present disclosure, the main control board is fixed on the main board frame; the main board frame is fixed on the head bracket.

In the present disclosure, the head mechanism is connected to the robot neck mechanism through bearings, and can also be adjusted through linkage devices, spherical devices, etc. during the specific implementation process.

In the present disclosure, the head rear shell is provided with a positioning portion that is adapted to the shape of the head bottom shell and the head DC motor and its transmission structure; the fixed part on the bottom shell of the head is fixed to the positioning part by screws through the screw fixing holes.

The present disclosure also proposes a neck mechanism for intelligent robots, comprising: a neck DC motor, a neck gear set, double-sided bearings, a neck bracket, and upper and lower shells; wherein,
the neck DC motor controls the neck mechanism to roll up and down through the neck gear set and double-sided bearings;
the upper and lower shells are divided into an upper half and a lower half, and the upper half and the lower half are fixed by screws;
the double-sided bearings on both sides of the neck mechanism are connected to the neck bracket, and the neck bracket is provided with a fixing part that can be fixedly connected to the robot body mechanism;
double-sided bearings are used to enhance the strength and stability of the mechanism, ensure smooth rotation and reduce noise;
the lower parts of the upper and lower shells are semicircular.

In the present disclosure, the maximum up and down nodding movement range of the neck mechanism is 90 degrees each.

In the present disclosure, the neck gear set comprises: a motor gear and a transmission gear; the motor gear is driven by the neck DC motor and meshes with the transmission gear; the transmission gear drives the connected double-sided bearings, and the double-sided bearings are fixed on the upper and lower shells to drive the movement of the upper and lower shells.

### Examples of Nodding Movements:

starting state: First, determine whether the robot's head, neck, and body are in the starting position, that is, the head, neck, and body are on the same axis, and the position of the company's logo on the body shell is preset as the starting position. If not in the starting position, the motor control board drives the head, neck, and body motors to move, so that the head, neck, and body return to the starting position.
nodding action:
1. Trigger the nodding action.
2. The head, neck and body are in the starting position.
3. According to the preset action duration and amplitude of the system, the motor control board adjusts the speed and angle of the neck DC motor to complete the up and down movement of the neck at the specified angle within the specified time. The time and angle values can be adjusted as needed.
4. The neck DC motor drives the motor gear, the motor gear drives the transmission gear, the transmission gear drives the connected double-sided bearings, and the double-sided bearings are fixed on the neck bracket, thereby driving the movement of the upper and lower shells.
5. The top of the neck mechanism is fixedly connected to the bottom of the robot head mechanism, and the movement of the neck drives the head to follow the movement.
6. Complete the nodding action.

In the present disclosure, the top of the neck mechanism is fixedly connected to the bottom of the robot head mechanism to realize the nodding action of the robot.

The present disclosure also proposes a body structure for an intelligent robot, comprising: a body shell, a turntable DC motor and its transmission structure, a speaker cavity, a turntable and a base body arranged in the body shell; wherein,
a body bearing assembly is fixed on the turntable;
the body shell is fixed on the turntable by screw connection, and it rotates with the turntable;
the turntable DC motor and its transmission structure comprise: a body bearing group, a body gear group and a turntable motor frame; wherein the turntable DC motor drives the body gear group to rotate, and the body gear group is connected to the body bearing group; the turntable DC motor controls the rotation of the body mechanism, and is fixed on the turntable motor frame; the turntable motor frame is fixed on the turntable;
the speaker cavity is fixed on the turntable.

In the present disclosure, the body bearing group comprises: an inner bearing and an outer bearing; wherein,
the inner bearing is directly connected to the body gear set and is fixed on the turntable to drive the turntable to rotate;
the outer bearing is fixed in a groove close to the outer side of the turntable, and the outer bearing supports the outer shell of the body without deformation.

In the present disclosure, a speaker hole is arranged on the body shell, which corresponds to the position of the speaker cavity. The body shell rotates together with the turntable to ensure that the speaker cavity always corresponds to the speaker hole.

In the present disclosure, the top of the body shell is in an inwardly concave arc shape, which is used to place the robot neck mechanism.

In the present disclosure, the rotation range of the body mechanism is 345 degrees.

In the present disclosure, the body shell is connected to the turntable and rotates with the turntable, and the maximum rotation range is determined by the limit point.

In the present disclosure, neck shaft fixing frames are arranged on both sides of the inner wall of the body shell, which are used to connect the neck mechanism of the robot.

In the present disclosure, the base body comprises: a base frame, a power control board and a rechargeable battery; the power control board is installed on the base frame, and a power input interface and a power output interface are arranged on the power control board;
the power input interface, the power output interface and the rechargeable battery are connected to the power control board through wires.

In the present disclosure, the turntable is fixed on the base frame of the base body by fixing screws.

In the present disclosure, the power control panel is further arranged with: a power switch and a network distribution button.

In the present disclosure, the turntable comprises: a turntable base and a motor control board.

Based on the head mechanism, neck mechanism, and body mechanism for an intelligent robot proposed above, the present disclosure further proposes an intelligent robot, comprising: a head mechanism, a neck mechanism, and a body mechanism; wherein,
the head mechanism comprises: a head bracket, a head body, a head shell, a display screen and a head bottom shell; the head body, the head shell, the display screen and the head bottom shell are all fixed on the head bracket; wherein,
the head shell comprises: a mask and a head back shell;
the head body comprises: a main control board, a capacitive touch sensor, a head DC motor and its transmission structure; the capacitive touch sensor is fixed in the head shell;
the head DC motor and its transmission structure comprises: a head DC motor, a head motor frame, a head gear set and a bearing; the head DC motor drives the head gear set to rotate, and the head gear set is connected to the bearing; the bearing is arranged on the head bottom shell; the head DC motor controls the shaking motion of the head mechanism, and is fixed in the head motor frame; the head motor frame is fixed on the head bottom shell;
the capacitive touch sensor communicates with the main control board to transmit touch sensing signals, and the main control board controls the movement of the head DC motor;
the display screen is arranged inside the head body, and the main control board is fixed at the back thereof;
the body mechanism is connected to the head mechanism through the neck mechanism.

In the intelligent robot of the present disclosure, the maximum rotation angle of the head mechanism to the left and right sides is 180 degrees. The neck mechanism can nod up and down with a maximum range of 30 degrees each.

The intelligent display processing terminal is detachable and can be carried and used alone;
the intelligent display processing terminal is capable of running system software, acquiring, saving and analyzing daily health monitoring data, communicating data with the cloud platform, and communicating with system hardware devices, etc.;

In order to make the intelligent display processing terminal have better stability in terms of software and hardware, the intelligent display processing terminal has the characteristics of being waterproof, dustproof, and drop-proof.

In a specific embodiment, the intelligent display processing terminal and the companion interactive intelligent robot realize the scenario-triggered interactive operations through WIFI, and the intelligent display processing terminal sends to the robot voice broadcast contents, including user health measurement data and home environment monitoring data content; and/or,
the data transmission between the intelligent display processing terminal and the multifunctional intelligent medicine box is transferred via WIFI using the health care cloud platform; and/or,
the intelligent display processing terminal and the third-party health monitoring hardware equipment realize data collection through WIFI/Bluetooth communication technology.

In specific embodiment methods, the system further comprises a base, and the base is provided with a companion interactive intelligent robot interface and/or an intelligent display processing terminal interface; the companion interactive intelligent robot can be rotatably fixed on the base through bolts, and the intelligent display processing terminal is fixed and/or charged to the base via magnetic attraction and/or of a base charging socket provided on the base; and/or,
the base is provided with a fitting base support with an adjustable angle and direction, and the intelligent display processing terminal can be fixed to the base via the fitting base support;
a back surface of the base is provided with a base power switch, the base power switch is used for controlling the on-and-off of the base and of the robot; and/or,
the base is provided with a base groove, a position on the intelligent display processing terminal in contact with the base via the base groove.

In specific embodiment methods, the base further comprises a robot control module and/or an Android development board and/or a base loudspeaker;
wherein the robot control module comprises a base ESP32 control module and/or a voice recognition module and/or a head liquid crystal screen control module and/or a motor driving chip;
the base ESP32 control module is used for implementing WIFI and Bluetooth functions;
the voice recognition module is used for recognizing a voice so as to implement an interaction with the robot, and playing back a synthesized voice;
the motor driving chip is used for driving a motor inside the robot to move;
the Android development board is used for providing a control function for the voice and/or the screen and/or the motor, and/or implementing WIFI and Bluetooth functions;
according to the requirements, the WIFI and Bluetooth communication functions in the ESP32 control module of the base can also be integrated into the Android development board, omitting the structure of the ESP32 control module of the base;
the base loudspeaker is used for playing back the synthesized voice.

The surface and internal structures of the base can be adjusted according to actual usage needs and scenarios to meet the corresponding usage requirements; In addition, some structures can also be added or deleted according to actual needs.

The base can be powered by an external circuit, and in special cases, it can also be powered by a battery with high energy density and high power output installed in the base.

The health care cloud platform used in the present disclosure is an integrated system platform capable of bi-directionally communicating with the family end and/or the service back end and/or the home intelligent end; the home intelligent end is in bi-directional communication with the intelligent medicine box end and/or the robot end;
the health care cloud platform is used for providing functions including health management and/or medication management and/or alarm management and/or report management and/or algorithm management and/or device linking management and/or user management and/or family management and/or operation monitoring and/or data management and control and/or message management and/or video calling;
the family end is mounted on a smart device and is used for implementing functions including linking with the user and/or receiving health reports of the user and/or video calling with the user;
the service back end is used for management of a system back end and access to a third-party service resource, and is capable of providing functions including call center calling and/or service allocation and/or service access;
the home intelligent end is configured to comprise a health management module and/or a medication management module and/or an exercise rehabilitation module and/or a sleep management module and/or a video calling module and/or a health report module and/or an emergency calling module and/or a device linking module, etc., and is used for implementing functions including health management and/or medication management and/or exercise rehabilitation and/or sleep management and/or video calling and/or health reporting and/or emergency calling and device linking;
the intelligent medicine box end communicates with the health care cloud platform, and is capable of acquiring medication information of the user, reminding the user of medicine types and/or dosages and/or medication precautions at set times and, after detecting that the user has taken medicine from the intelligent medicine box, synchronizing information to the health care cloud platform, so as to implement functions including medication reminding and medication statistics;
and the robot end communicates with the health care cloud platform, and is capable of triggering interaction with the user under a preset specified environment, and executing a corresponding action and expression instruction according to an interactive scenario, so as to implement functions including voice recognition and/or situational dialogue and/or care reminding and/or action and expression presentation.

The smart health care system is further capable of importing into the smart health care system data in an external access device by means of a device access protocol.

The intelligent health care system in the present disclosure can achieve functions including but not limited to user intelligent health care technology, medicine box design and medication management, companion robot design, home environment monitoring, etc.

The present disclosure also provides the application of the companion interactive intelligent health care system in scenarios including health monitoring and management, medication management, interactive companionship, environmental monitoring and early warning.

Based on the above system, the present disclosure also proposes an intelligent medication management method, comprising the following steps:
step 1: setting medication information in the intelligent display processing terminal, sending instructions to the companion interactive intelligent robot through the cloud platform, and when reaching a medication time, playing by the companion interactive intelligent robot a voice reminder to the user to take the medicine, receiving voice feedback from the user on taking the medicine or not, and stop playing the voice reminder;
step 2: when a preset medication time has come, playing the name and dosage information of the medicine by the medicine box, lighting up the screen to display the name and dosage information of the medicine, and turning on a medicine box status LED light at the front of a medicine storage drawer of a medicine storage box of the medicine box to reminder the user to open the drawer and take the medicine;
step 3: after the medicine storage drawer is opened, turning on a partition board LED button light of a medicine compartment, and after the medicine is taken and put back, closing the medicine storage drawer when the partition board LED button light is pressed, turning off the partition board LED button light and the medicine box status LED light to indicate that the medicine has been taken on time, sending a signal to the cloud platform to record the medication situation, and generating a medication report.

In the present disclosure, the medication report of the user is remotely viewed through an intelligent medicine box child-side application of the intelligent display processing terminal.

In the present disclosure, during medication, a function button of a machine head below a display screen can be long pressed to stop the voice playing.

In the present disclosure, when the medicine storage drawer is not opened at the time of taking the medicine, the medicine box will repeatedly remind to take the medicine 3 times by voice, and after an interval of 2 minutes, remind again 3 times, repeat 5 times, and then stop broadcasting; if the on-time medication signal is still not received after 30 minutes, the cloud platform will send an early warning message to the child-side application and initiate human intervention measures.

In the present disclosure, a camera on the medicine box is configured to scan medicine barcodes to query the medication information. After scanning the barcode on the medicine package, the intelligent medicine box transmits the medicine barcode information to the cloud platform via WIFI to query the medicine information; if the medicine information can be queried, check whether the medication plan has been set.

The steps for querying the medicine information are as follows:
1. Press and hold the function key to activate the camera: The user presses and holds the function key to activate the device's camera.
2. Scan the barcode: The user scans the medicine's barcode using the activated camera.
3. Determine whether the medicine information is found:

If medicine information is not found, the system would provide a voice broadcast or display on the screen: “Medicine information not found. If you need to set up a medication plan, please manually add it.” At this point, the process moves to the medication setup stage.

If medicine information is found, the system proceeds to the next step.
4. Determine whether the medication plan is already set:

If no medication plan has been set, the system would provide a voice broadcast or display on the screen: “The medicine name, indications, and intervals all need to have a medication plan set.” The user can then proceed to set up the medication plan.

If the medication plan has been already set, the system would provide a voice broadcast or display the medicine name, dosage, and medication plan information.
5. Complete the medicine information inquiry: Once the medicine information and medication plan are confirmed, the medicine information inquiry process is completed, and the next stage of setting up the medication proceeds.

Further, if the medicine information can be found and the medication plan has been set, the cloud platform will send the medicine information to the intelligent medicine box and inform the medicine information and medication plan through voice broadcast and screen display;
if the medicine information can be found and there is no medication plan, the cloud platform sends the medicine information to the intelligent medicine box, which broadcasts the medicine information by voice and displays it on the screen, and then asks by voice and on the screen whether a medication plan needs to be set. After receiving the user's voice and screen feedback, if a medication plan needs to be set, the user enters the medication setting. If a medication plan does not need to be set, the voice broadcast and screen display are completed, and the user only needs to query the medicine information;
if no medicine information is found and a medication plan needs to be set up, enter the medicine name, dosage, and medication time through voice or screen input;
if the medicine information is not found and there is no need to set up a medication plan, the voice broadcast and screen display will indicate that the medicine information is not found.

Further, if the medicine information can be found and the medication plan has been set, the cloud platform will send the medicine information and medication plan to the intelligent medicine box; the medicine box will broadcast the medicine information by voice and display the medication plan on the screen; the LED indicator on the front panel of the medicine box drawer will light up; open the medicine box, the corresponding medicine compartment LED indicator will light up, put the medicine in the designated medicine compartment with or without packaging, and press the medicine compartment LED button to indicate that the medicine has been put in;
if the medicine information cannot be found, the cloud platform will feed back the query results to the intelligent medicine box via WIFI, and voice broadcast and screen display will ask whether a medication plan needs to be set up; when the reply is that medication needs to be set up, activate the voice input and screen manual input functions, enter the medicine name, medication time and dosage; the indicator light of the intelligent medicine box lights up, open the medicine storage drawer, select the medicine compartment to put the medicine, press the medicine compartment LED button, and the medicine compartment information is sent to the medicine box control terminal via Bluetooth. The intelligent medicine box uploads the medication information and medicine compartment location information to the cloud platform via WIFI;
if the medicine information can be queried, but there is no record of medication settings, it means that the medicine is entered for the first time; the cloud platform sends the medicine information back to the intelligent medicine box via WIFI, and the intelligent medicine box asks whether a medication plan needs to be set up through voice broadcast and screen display; when the reply is that medication needs to be set up, the system automatically enters the medicine name information, and activates the voice entry and screen manual input functions at the same time. The user only needs to set the medication time and dosage.

In the present disclosure, the intelligent medicine box with an expanded structure comprises: a machine head, at least one medicine storage box;
the machine head is a medicine box display and control terminal, comprising a machine head body and a machine head base; the machine head body and the machine head base are integrated structures;
the medicine storage box comprises: a box shell and a medicine storage drawer, wherein the medicine storage drawer can be pulled out and moved inside the box shell;
the machine head matches the top structure of the medicine storage box, so that the machine head is fixedly connected to the medicine storage box.

A U-shaped slide rail structure and a positioning slot are arranged on the top of the shell of the medicine storage box, and a power supply pin is arranged at the bottom of the U-shaped slide rail structure on the top of the medicine storage box; a T-shaped structure base is arranged at the bottom of the shell of the medicine storage box, and a box installation buckle is arranged at the bottom of the T-shaped structure base, and a box bottom pin is arranged at the rear of the T-shaped structure base; the T-shaped structure base corresponds to the U-shaped slide rail structure, the box bottom pin contacts with the power supply pin at the bottom of the U-shaped slide rail structure on the top of other medicine storage boxes to supply power to other medicine storage boxes.

The rear portion of the box shell is provided with a box bottom pin, a power distribution board, and a Hall induction magnet.

The box bottom pin is arranged at the lower part of the rear part of the box shell; the power distribution board includes three circuit interfaces, one of which inputs a power supply signal from the power supply pin at the top of the medicine storage box, and the other two are respectively connected to the pins at the bottom of the box through wires, and connected to a Bluetooth main control board on the medicine storage drawer through a spring power line, respectively supplying power to the medicine storage box and the Bluetooth main control board; the Hall sensor magnet corresponds to the position of the Hall sensor board arranged on the medicine storage drawer.

The robot includes the following functions:
companionship interaction, daily life information query and broadcast, user health information query and broadcast, medication information reminder query and broadcast, and remote interactive calls with family members; wherein,
the companion interaction includes: scenes, voices, expressions, and actions, which are superimposed to generate interactive effects;
the daily life information query and broadcast is: accessing the cloud platform through WIFI, broadcasting daily life information based on user query needs;
the user health information query and broadcast is: access the robot's exclusive cloud platform through WIFI, associate the application APP, and broadcast daily health information based on user query needs;
the medication information query and broadcast are as follows: access the robot's exclusive cloud platform via WIFI, associate the application APP, and regularly broadcast reminders for users to take medication; query medication at any time, and broadcast medication information based on user needs.

In the present disclosure, the companionship interaction also includes but is not limited to the following specific scene actions:
1) Head shaking motion: There is one capacitive touch sensor on the top of the robot's head and two on both sides of its cheeks. When the user touches the robot's head and cheeks, the robot turns on the motors of its head and neck and shakes its head;
2) Emotional outbursts: The speed and amplitude of various movements of the body, head, and neck are amplified; in scenarios where executive function fails, including speech recognition failure, etc.;

### Examples of Emotional Outbursts:

starting state: No need to determine whether it is at the starting position, take the current position as the starting state.
emotional outbursts:
1. Trigger emotional outburst actions.
2. The motor drive board drives the head, neck, and body motors to move simultaneously. The head and body rotate rapidly left and right in opposite directions, while the neck moves rapidly up and down. The motor speed can be adjusted as needed.
3. According to the system preset action duration, amplitude and number of repetitions, the motor drive board adjusts the motor speed and angle of the head, neck and body to complete the specified angle movement within the specified time and repeat it according to the preset number of repetitions. The time and angle value settings can be adjusted as needed.
4. Complete the emotional outburst actions.

In the present disclosure, the robot, the application APP and the cloud platform are interconnected to complete the background data exchange; the information setting is completed on the application APP of the mobile terminal; the cloud platform is used as the information transfer center, the application setting information is synchronized with the cloud platform, and then sent to the intelligent robot by the cloud platform; the intelligent robot feeds back the user interaction information to the cloud platform, which is then synchronized to the application by the cloud platform; the intelligent robot needs to access the network through Bluetooth and is set in the application APP.

In the present disclosure, in the medication reminder broadcast,
the medication information of the intelligent robot is set on the APP side, and the instructions are sent to the intelligent robot through the cloud platform. When the medication time has come, the intelligent robot would announce the name of the medicine by voice to remind the user to take the medicine on time and in the right amount. The user would be prompted to give feedback through voice on whether to take the medicine. and the voice announcement would stop.

The intelligent robot only has the function of timing reminders and has no judgment function to prevent misleading users.

In the present disclosure, the medication reminder broadcast includes the following steps:
1) Users set medication information in the APP and synchronize it to the cloud platform;
2) The cloud platform issues instructions to inform the intelligent robot of the time and medication information to be broadcasted for each medication reminder set by the user;
3) The intelligent robot determines the arrival time of medication based on the pre-stored medication time and system time calibration;
4) The intelligent robot broadcasts medication information, i.e. medication reminders, medicine names, and dosages; at the same time, the intelligent robot locally triggers the screen to display medication reminder expressions, and the robot performs medication reminder supporting actions;
5) Play the medication reminder voice according to the preset number of repetitions and time intervals, turn off the screen after the broadcast, and wait for the user to feedback on medication information;

If the medication is taken on time, the user will inform the intelligent robot by voice;
6) Intelligent robots trigger “voice+action+expression to take medicine on time”;
7) Turn off the screen;
If the user does not tell the robot whether to take the medicine on time;
8) If the robot does not receive feedback from the user that the medication was taken on time after the preset time, it returns to step 4;
9) After repeating step 4 for the preset number of times, the robot still does not receive the instruction from the cloud that the medication has been taken on time;
10) Intelligent robot triggers “voice+action+expression for not taking medicine on time”;
11) Stop the announcement and turn off the screen.

In the present disclosure, in the health status query and broadcast, the daily health monitoring data of the intelligent robot is input on the application APP side, and the data is synchronized to the cloud platform. When the user inputs the query requirement through voice, the intelligent robot sends a query request to the cloud platform, and the cloud platform sends the health information to the intelligent robot, and the intelligent robot broadcasts the health status information through voice.

In the present disclosure, in the daily communication and interactive companionship,
the user wakes up the intelligent robot by voice, and the intelligent robot turns its body to face the user through sound source and visual positioning, and broadcasts the corresponding voice, expression, and action at the same time; the user sends voice to the intelligent robot, the intelligent robot receives the user's voice information and sends it to the cloud. The cloud parses the user's voice commands, makes different responses according to the user's commands, and sends commands to the robot to execute the corresponding voice, expression, and action.

In the present disclosure, the daily communication and interactive companionship includes the following steps:
1) The user wakes up the intelligent robot through the wake-up word; the wake-up word is recognized locally and the corresponding wake-up operation is performed:
action+expression+voice;
expression: local wake-up expression;
voice: Local built-in voice;
action: The body and head are on one axis, and the head returns to a 90-degree position with the main axis;
2) After the intelligent robot receives the wake-up command locally, it uses sound source and visual positioning to turn its body to face the user, while broadcasting the corresponding voice, expression, and action;
3) The user sends a voice to the intelligent robot;
4) The intelligent robot receives the user's voice and uploads it to the cloud platform;
5) After analyzing the user's voice information, the cloud platform determines whether the user's voice is a query or daily communication;
6) The cloud platform generates answers based on user query information and synthesizes corresponding voice feedback information;
7) The cloud platform sends the answering voice to the intelligent robot, and the command triggers the corresponding expression and action;
8) The intelligent robot plays the voice sent by the cloud platform and triggers corresponding expressions and actions;

If the user's voice is a query command;
9) Turn off the screen after the announcement is completed; If the user's voice is for daily communication;
9) After the announcement is completed, wait for the user to continue to interact. If there is a new user interaction within the preset time, repeat step 9;
10) After the announcement is completed, if there is no new interaction within the preset time, the intelligent robot will turn off the screen.

The innovative features of the intelligent robot of the present disclosure include:
1. The hemispherical neck structure design has no similar structure used for desktop robots in the current domestic market; this design improves the load-bearing capacity and increases movement flexibility.
2. The body rotation adopts an inner and outer bearing structure. The inner bearing is connected to the gear and motor, and the outer bearing is connected to the outer shell of the robot body. The outer bearing is also called a plane bearing, which can not only make the body rotate smoothly, but also support the outer shell to prevent deformation.
3. Combine display, voice, and motor motion functions to enable robots to interact with people, keep in touch with family members, and accompany daily life.

Based on the above method, the present disclosure also proposes a medication reminder system based on a multifunctional intelligent medicine box, the system comprising:
a request receiving module, used to receive the medicine placing, taking and taking data sent by the smart medicine box;
a medicine information query module, used to query medicine information;
a data matching module, used for matching the user's medication plan according to the medicine information;
a plan generation module, used to generate a medication plan when there is no matching medication plan for the medicine information;
a plan push module, used to send the medication plan reminder to the user terminal, and the medication plan reminder is used to instruct the intelligent medicine box to remind the user to take the medicine;
a storage module, used for storing medicine information and medication plans;
a request sending module, used to send the medication plan and medication records to the cloud server, and the child-side application can remotely view the medication records.

The beneficial effects of the present disclosure include:
assist the user in taking medicines on time and in fixed quantities. The intelligent medicine box will remind the user to take medicines by announcing the name and dosage of the medicines at the designated time, and the screen will display the name and dosage of the medicines. This will prevent the user from taking multiple medicines at the same time and causing adverse medicine reactions and medicine interactions due to missed medication times and incorrect dosages, which will affect the treatment effect and quality of life. Ensure that the user takes medicines correctly to control the development and deterioration of chronic diseases and reduce the occurrence of complications.

The intelligent medicine box accurately locates the storage location of medicines, and responds with multiple functions such as LED lights, voice broadcasts, screen displays, etc. to help the user take medicines in the correct medicine compartment. There are multiple compartments in the medicine box for storing different types of medicines. The smart medicine box is equipped with LED light buttons in each compartment. When the time to take medicine is up, the user opens the medicine box, and the indicator light on the corresponding compartment lights up and flashes, reminding the user to take out the correct medicine from the medicine compartment, avoiding the user taking out the wrong medicine, resulting in medication errors and adverse medicine reactions, causing adverse consequences.

The intelligent medicine box has a WIFI function, which can help the relatives of the user to remotely understand the user's medication situation and the status of the medicine box equipment. When the user does not take the medicine on time and in the prescribed amount, the system would automatically send an alarm message to the relatives of the user, and can manually intervene in the rational use of the user. When the battery of the medicine box is too low, the power plug is loose, the WIFI connection is not smooth, etc., the cloud platform will send an alarm message to the relatives to help the user solve the equipment problem in time.

When the user needs to take multiple medicines and one medicine box cannot meet the needs of storing all the medicines, multiple medicine boxes can be stacked to expand the storage space for medicines without changing the head. Multiple medicine boxes are stacked together through a mechanical structure and connected to the power supply through the pin structure at the end of the medicine box. Multiple medicine boxes can be numbered manually or automatically by Bluetooth and controlled by the same machine head. It meets the needs of the user to store multiple medicines and saves storage space and costs.

The present disclosure provides a multifunctional intelligent robot device that can coordinate and interact with scenes, voices, movements, and expressions and can talk with family members remotely.

The robot of the present disclosure obtains the medication information of the user through the associated APP, and reminds the user to take the medicine on time and in the prescribed amount by superimposing voice, expression and action. In daily life, the user can also check their medication information at any time by voice.

The robot application APP of the present disclosure uniformly records the daily health data of the user. The user and the intelligent robot can retrieve and understand their health conditions at any time through voice, and relatives can also remotely check the health data of the user through the application APP, providing assistance for healthy user care.

### BRIEF DESCRIPTION OF THE DRAWINGS

FIG. 1 is a front view and a side view of the structure of the intelligent medicine box of the present disclosure.

FIG. 2 is a three-dimensional structural diagram of the intelligent medicine box of the present disclosure.

FIG. 3 is a structural diagram of a medicine storage drawer in the intelligent medicine box of the present disclosure.

FIG. 4 is a structural diagram of the head of the intelligent medicine box of the present disclosure.

FIG. 5 is a structural diagram of the head of the intelligent medicine box of the present disclosure from another angle.

FIG. 6 is a schematic diagram of the internal structure of a head in the intelligent medicine box of the present disclosure.

FIG. 7 is another schematic diagram of the internal structure of the head of the intelligent medicine box of the present disclosure.

FIG. 8 is yet another schematic diagram of the internal structure of the head of the intelligent medicine box of the present disclosure.

FIG. 9 is a schematic diagram of the back structure of the intelligent medicine box of the present disclosure.

FIG. 10 is a three-dimensional structural diagram of the medicine storage box in the intelligent medicine box of the present disclosure.

FIG. 11 is a rear structural diagram of the medicine storage drawer in the intelligent medicine box of the present disclosure.

FIG. 12 is a schematic diagram of the structure of the bottom of the intelligent medicine box of the present disclosure.

FIG. 13 is a schematic diagram of the use status of the intelligent medicine box of the present disclosure.

FIG. 14 is a schematic diagram of the structure of the back panel of the intelligent medicine box drawer of the present disclosure.

FIG. 15 is a schematic diagram of a medication reminder system based on a multifunctional intelligent medicine box according to the present disclosure.

FIG. 16 is a schematic diagram of the robot head mechanism with a mask.

FIG. 17 is a schematic diagram of the robot head mechanism without a mask.

FIG. 18 is a schematic diagram of the structure of the head mechanism with the head DC motor inside.

FIG. 19 is a side view of the interior of the head mechanism.

FIG. 20 is a rear view of the interior of the head mechanism.

FIG. 21 is a stereoscopic view of the interior of the head mechanism.

FIG. 22 is a front structure of the head mechanism without a mask.

FIG. 23 is a schematic diagram of the patch capacitive touch sensors and positioning part in the rear shell of the head.

FIG. 24 is a schematic diagram of the head bearing of the head mechanism.

FIG. 25 is a side view of the head mechanism.

FIG. 26 is a rear view of the head mechanism.

FIG. 27 is a schematic diagram of the neck mechanism including the neck DC motor.

FIG. 28 is an exploded view of the head mechanism.

FIG. 29 is an exploded view of the neck mechanism.

FIG. 30 is a front view of the neck mechanism.

FIG. 31 is a stereoscopic view of the neck mechanism.

FIG. 32 is a side view of the neck mechanism.

FIG. 33 is a bottom view of the neck mechanism.

FIG. 34 is a top view of the neck mechanism.

FIG. 35 is a diagram of the body mechanism showing neck shaft fixing frames.

FIG. 36 is a schematic diagram of the transmission structure of the turntable part of the body mechanism.

FIG. 37 is a top view of the body mechanism.

FIG. 38 is a bottom view of the body mechanism.

FIG. 39 is a side view of the body mechanism.

FIG. 40 is a rear view of the body mechanism.

FIG. 41 is a schematic diagram of the transmission structure of the turntable part of the body mechanism with neck shaft fixing frames.

FIG. 42 is the turntable DC motor and its transmission structure, the motor control board, and the speaker cavity in the body mechanism.

FIG. 43 is a front view of the body mechanism.

FIG. 44 is a schematic diagram of a base body with a base frame, a power control board and a turntable base.

FIG. 45 is a schematic diagram of the structure of the base frame.

FIG. 46 is a schematic diagram of the main structure of the robot.

FIG. 47 is an exploded view of the body structure.

FIG. 48 is a relationship diagram of the robot, cloud platform, and application program of the present disclosure.

FIG. 49 is a three-dimensional image of the robot from one angle.

FIG. 50 a three-dimensional image of the robot from another angle.

### DETAILED DESCRIPTION

The multifunctional intelligent medicine box proposed by the present disclosure is an intelligent medication management product that combines voice, display, WIFI access to cloud platforms, expandable structure, and locatable medicine storage locations. It meets the needs of hearing, vision, remote monitoring, storage, and management of medication, and is suitable for different scenarios and populations.

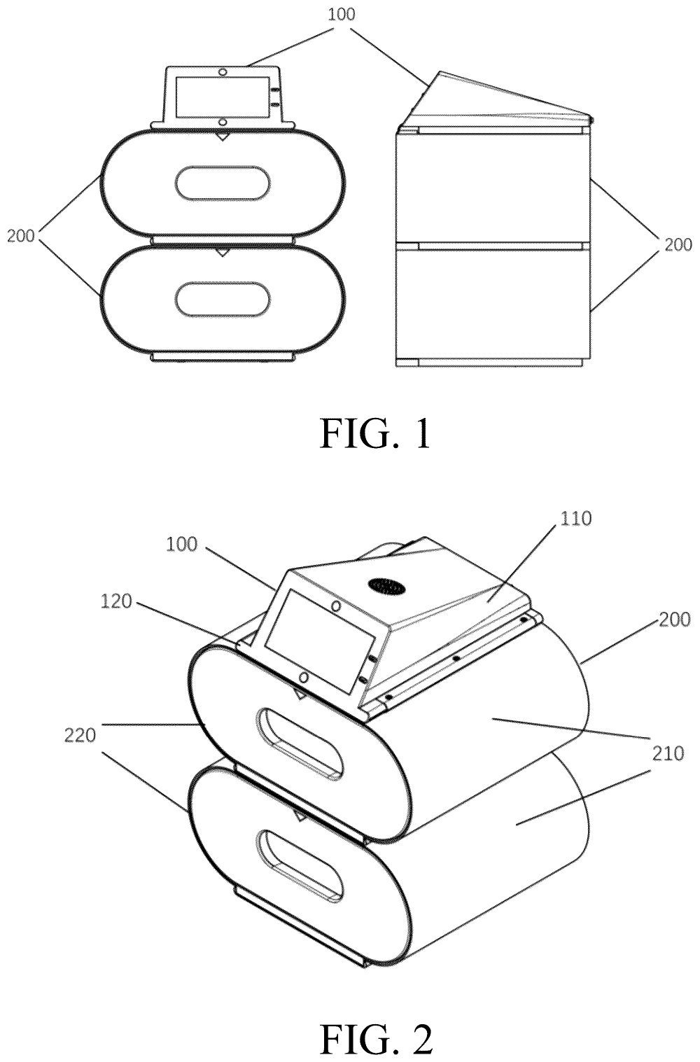

The present disclosure provides a multifunctional intelligent medicine box with an expanded structure, the multifunctional intelligent medicine box comprising: a machine head 100, and at least one medicine storage box 200, as shown in FIG. 1;
as shown in FIG. 2, the machine head 100 is a medicine box display and control terminal, comprising a machine head body 110 and a machine head base 120; the machine head body 110 and the machine head base 120 are integrated structures;
the medicine storage box 200 comprises: a box shell 210 and a medicine storage drawer 220, wherein the medicine storage drawer 220 can be pulled out and moved inside the box shell 210;
the machine head 100 matches the top structure of the medicine storage box 200, so that the machine head 100 can be fixedly connected to the medicine storage box 200.

The machine head base 120 is a T-shaped structure, which matches the U-shaped slide rail structure protruding from the top of the medicine storage box 200, and the machine head base 120 is inserted into the U-shaped slide rail structure 211 at the top of the medicine storage box 200 through the positioning buckle at the bottom of the machine head base 120 and the positioning slot at the top of the medicine storage box 200; the front end of the U-shaped slide rail structure 211 is equipped with a tilt angle structure, which facilitates the stacking and fixation of the machine head 100 and multiple medicine boxes 200.

The two sides of the T-shaped structure of the machine head base 120 can slide and snap into the inner side of the slide rail of the U-shaped slide rail structure 211 at the top of the medicine storage box 200; the tail of the machine head base 120 is a step-shaped structure, which matches and snaps with the bottom shape of the U-shaped slide rail structure 211 at the top of the medicine storage box 200; the positioning buckle 121 at the bottom of the machine head base 120 corresponds to the position of the positioning slot 212 at the top of the medicine storage box 200, and when the machine head 100 is fixedly connected to the medicine storage box 200, the positioning buckle 121 snaps into the positioning slot 212;
as shown in FIG. 10, the step-shaped structure of the machine head base 120 is provided with a pin interface 122 which contacts a power supply pin 213 at the bottom of the U-shaped slide rail structure 211 at the top of the medicine storage box 200 to supply power to the medicine storage box 200; the pin interface 122 of the machine head is connected to a power control board 140 via a wire.

As shown in FIG. 4, the front of the head body 110 is provided with a display screen 111, a scanning camera 112, function buttons 113, and a volume adjustment button;
the volume adjustment button comprises a volume up button 114 and a volume down button 115;
the scanning camera 112, the function buttons 113, and the volume adjustment buttons are distributed around the display screen 111;
a speaker hole 116 is arranged on the back of the machine head body 110.

In a specific embodiment, the display screen 111 can display the medicine name, dosage, current time, battery level, charging mode, WIFI connection status, and Bluetooth connection status;
the scanning camera 112 is arranged above the display screen 111, and can scan the barcode of the medicine package to obtain medicine information for medication management. For example, the medicine placed can be directly scanned, the barcode information is transmitted via WIFI, the medicine information is queried through the cloud platform, and the medicine information is voice broadcast and displayed on the display screen;
the volume adjustment button is arranged on the right side of the display screen 111 and is used to adjust the voice broadcast volume;
the function button 113 is set below the display screen 111, which can turn on/off the voice broadcast; long press to activate the camera scanning function, scan the barcode on the medicine package, and obtain medicine information. The function of the function button 113 can also be expanded to interrupt, start, confirm and other functions, including: 1. confirm the completion of the medication process on time; 2. confirm the completion of scanning the code to identify the medicine.

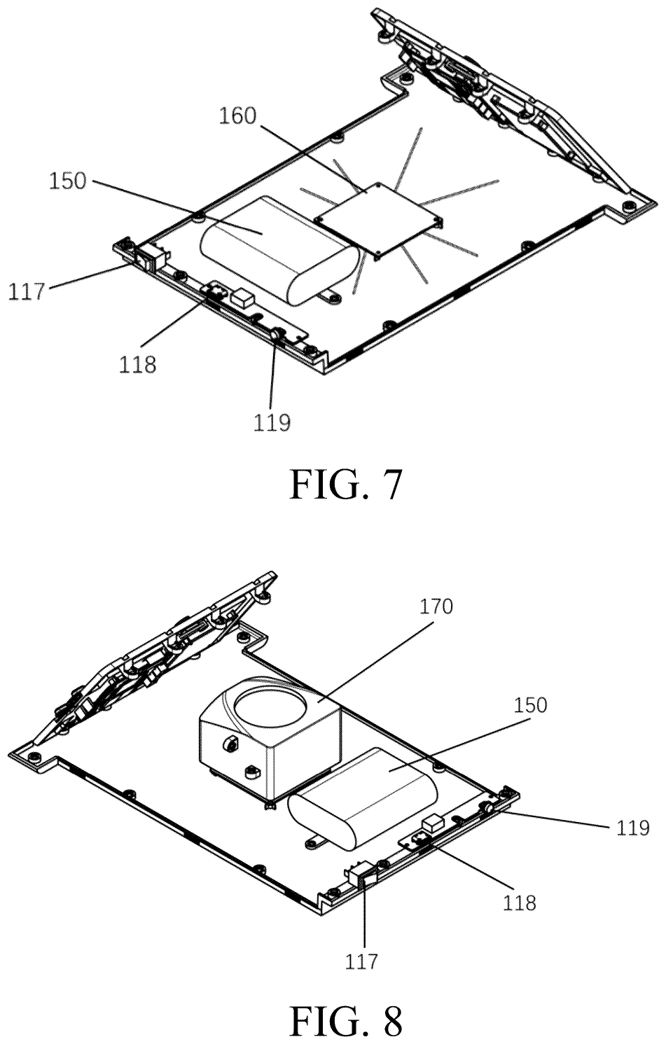

A control mainboard 130 and the power control board 140 are fixedly arranged at the back of the display screen inside the machine head body 110; the control mainboard 130 comprises a voice module, a display module, and a WIFI module;
the voice module is used to recognize and generate sounds; specifically, to regularly remind users to take medications, play the name and quantity of medications, and recognize user voice commands, including setting medication information, dosage, and time;
the display module is used to display the status information of the medicine box device, medication-related information and input medication information; specifically, it is used to control the display content of the machine head display screen 111, including medication-related information, such as the name of the medicine, the dosage; the location information of the medicine compartment where the medicine is stored; and the device information, including the current time, battery circuit, and network connection status;
the WIFI module is used to transmit device information and medication management information to the cloud platform; specifically, it is used to connect the medicine box and the cloud platform to transmit medication setting information and feedback on medication usage information;
the power control board 140 can serve as a transfer structure for electrical connection and can monitor battery power, power connection status, and machine switch status;
as shown in FIG. 7, a lithium battery 150, a Bluetooth antenna array board 160, and a speaker cavity 170 are fixedly arranged at the bottom of the interior of the machine head body 110; the speaker cavity 170 is installed above the Bluetooth antenna array board 160 and corresponds to the position of the speaker hole 116 on the back of the machine head body 110, as shown in FIG. 8;
the lithium battery 150 is electrically connected to the power control board 140 and can supply power to the machine head 100 and/or the medicine storage box 200; in particular, when the external power supply is cut off, the lithium battery 150 is the power source for the entire medicine box; the Bluetooth antenna array board 160 is used to transmit Bluetooth communication signals to achieve Bluetooth communication between the machine head 100 and the medicine storage box 200. Specifically, the Bluetooth antenna array board 160 can be used to communicate with the Bluetooth control board (Bluetooth microcontroller) on the partition board inside the medicine storage box 200; the speaker cavity 170 is used to play voice signals externally.

The LED light status signal and the button status signal are sent to the Bluetooth control board through wires, and the Bluetooth control board sends the medicine compartment position signal to the Bluetooth antenna array board 160 of the machine head through Bluetooth, and the Bluetooth antenna array board 160 sends the signal to the main control board through wires, and the main control board sends the position signal to the cloud platform through WIFI.

The control mainboard 130, the power control board 140, the lithium battery 150, the Bluetooth antenna array board 160, and the speaker cavity 170 are electrically connected, and the processed signals transmitted between them are aggregated to the control mainboard 130;
in a specific embodiment, the speaker cavity 170 is used to play voice signals, and the sound is emitted from the speaker hole 116 on the back of the head body;
the lithium battery 150 uses three 18650 lithium batteries, 2000 mA/cell, totaling 6000 mA;
the lithium battery 150 is three packaged lithium batteries provided by the manufacturer; the lithium battery 150 is connected to a power control board 140, and the power control board 140 can detect the battery power and issue an alarm when the power is lower than a threshold; the lithium battery 150 can also be selected according to actual needs.

The control mainboard 130 is RV1106; the control mainboard 130 can also be selected according to actual needs.

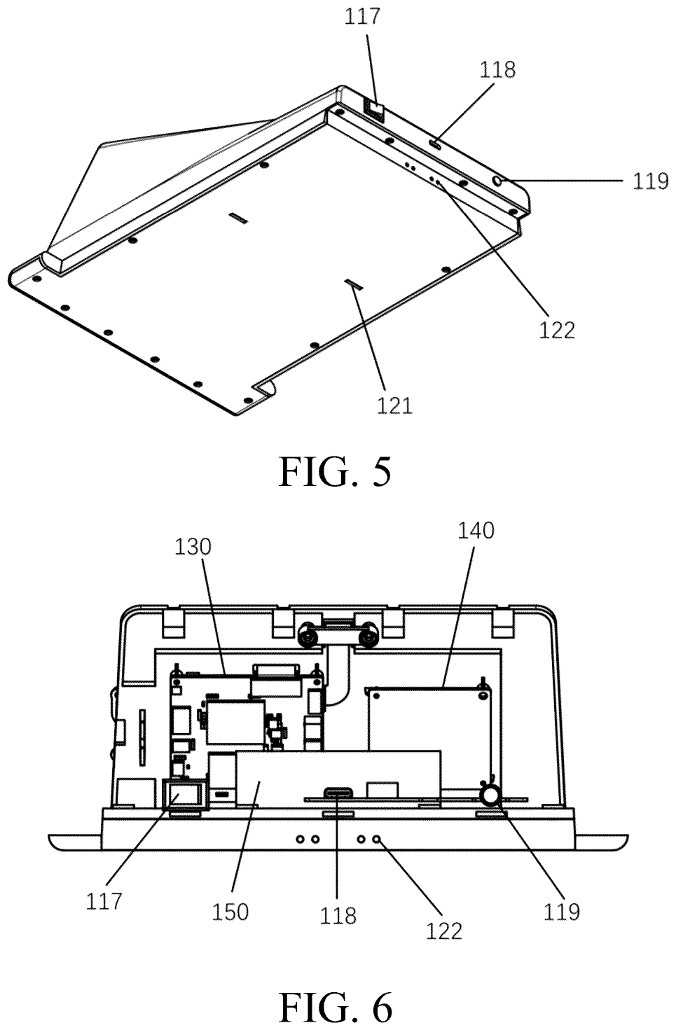

The lower side of the back of the machine head body 110 is also provided with a power switch 117, a power interface 118, and a network distribution button 119, as shown in FIG. 5;
the power switch 117 is electrically connected to the power control board 140, and can be used to control the start and shut down of the machine head 100 and/or the medicine storage box 200 by turning the power on and off;
the power interface 118 is electrically connected to the power control board 140 and is used to connect external wires to directly power the machine head 100 and/or charge the lithium battery 150 in the machine head 100, as shown in FIG. 6;
the network distribution button 119 is electrically connected to the control mainboard 130 and is used to connect to the WIFI.

In a specific embodiment, the power interface 118 is a Type-C interface.

A U-shaped slide rail structure 211 and a positioning slot 212 are arranged on the top of the shell of the medicine storage box 200, and a power supply pin 213 is arranged at the bottom of the U-shaped slide rail structure 211 on the top of the medicine storage box 200; as shown in FIG. 12, a T-shaped structure base is arranged at the bottom of the shell of the medicine storage box 200, and a box installation buckle is arranged at the bottom of the T-shaped structure base, and a box bottom pin 214 is arranged at the rear of the T-shaped structure base; the T-shaped structure base corresponds to the U-shaped slide rail structure 211, when there are multiple medicine storage boxes 200, due to the same mechanical structure of the upper and lower parts of the multiple medicine storage boxes 200, and the box bottom pin 214 contacts with the power supply pin 213 at the bottom of the U-shaped slide rail structure 211 on the top of other medicine storage boxes 200 to supply power to other medicine storage boxes 200.

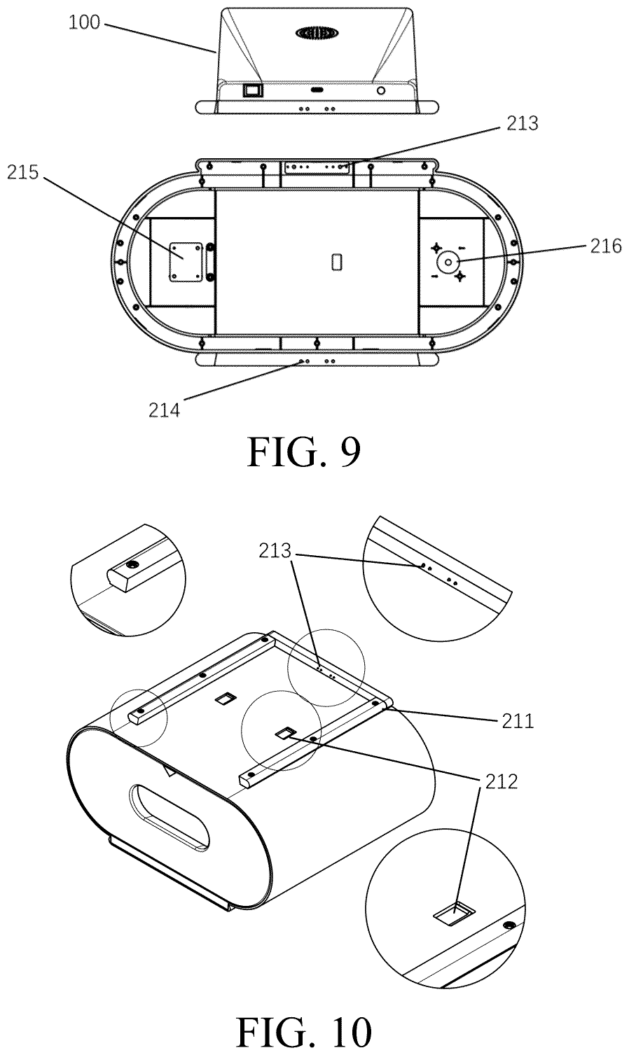

The rear portion of the box shell 210 is provided with a box bottom pin 214, a power distribution board 215, and a Hall sensor magnet 216, as shown in FIG. 9.

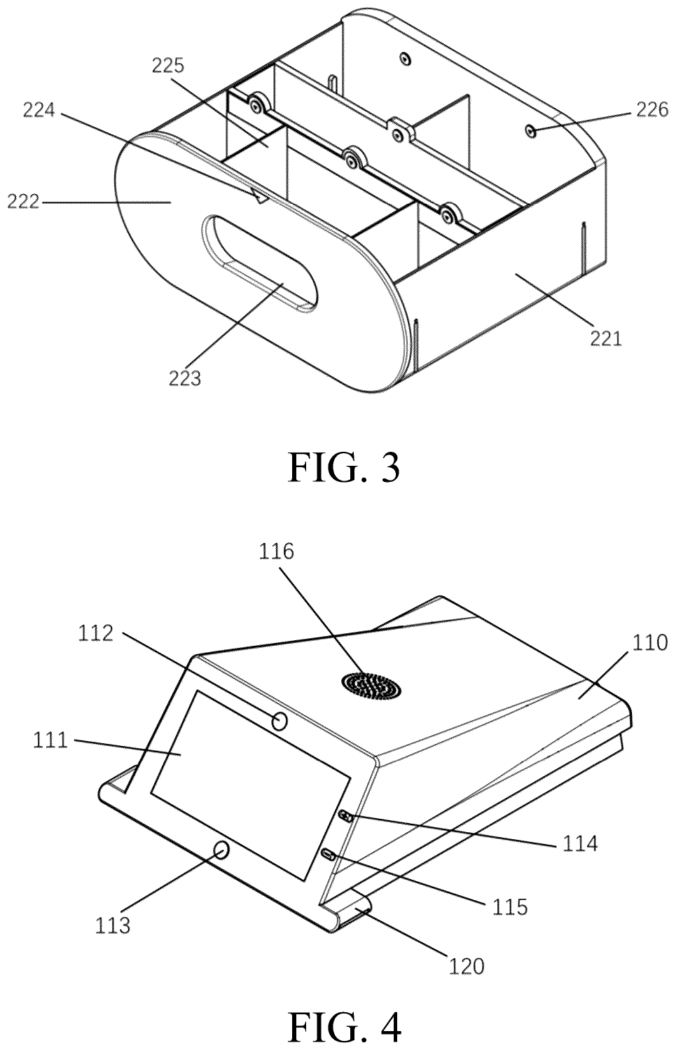

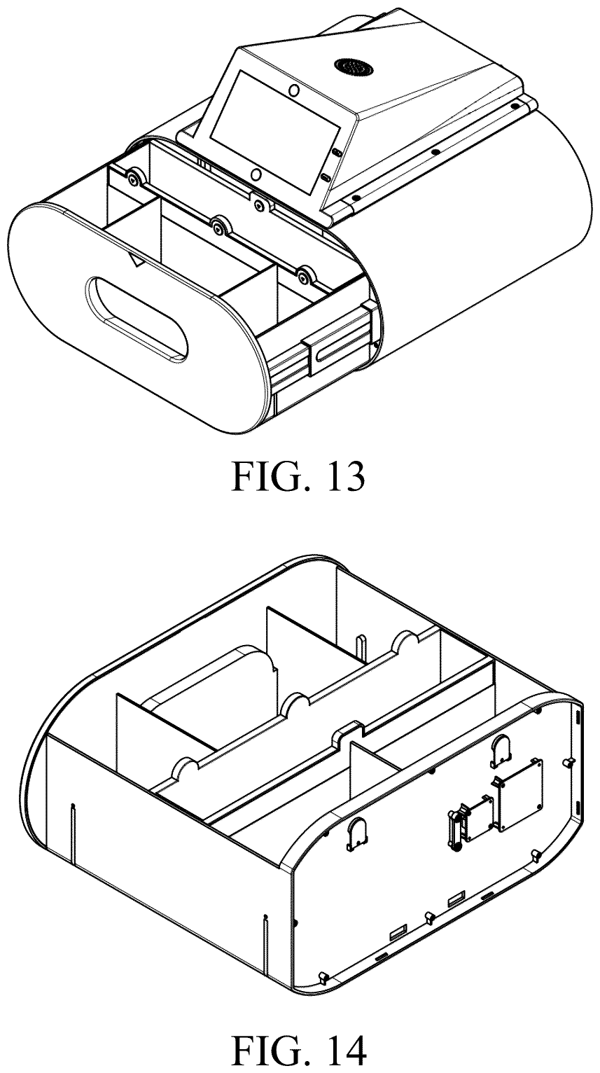

As shown in FIG. 3, the medicine storage drawer 220 comprises: a medicine storage drawer body; the medicine storage drawer body comprises: a front panel 222 and a drawer body 221; the front panel 222 is installed at the front end of the drawer body 221;
the front panel 222 is provided with a drawer handle 223, and the side surface of the drawer body is provided with a sliding groove; the sliding groove corresponds to a box slide rail provided inside the box shell 210, as shown in FIG. 13;
a medicine box status LED light 224 is provided at the top of the front panel 222, and the medicine box status LED light 224 can display different colors according to different scenes; it is off under normal circumstances, and is always green when taking medicine, and it is red when the medicine box drawer is not opened or closed properly; the color and status of the LED light are adjustable and are not limited to red, yellow and green. When there are multiple layers of medicine boxes, the medicine box status LED light 224 lights up to indicate the corresponding medicine box storing the medicine; for example, when it is time to take medicine, the LED light on the front panel 222 of the medicine box storing the medicine in the multiple layers of medicine boxes will light up, helping users to accurately find the location of the medicine box where the medicine is stored.

Inside the medicine storage drawer body is divided into medicine compartments of different sizes by pluggable partition boards 225, which can be used to store packaged medicines of different sizes, and the size of the medicine compartment can be customized according to actual needs; a protrusion is provided on the upper part of the partition board of each medicine compartment, and a partition board LED button light 226 is provided at the position of the protrusion; after putting in and taking out medicines, the partition board LED button light 226 can be manually pressed, the partition board LED button light 226 lights up, and the position signal is sent to the control mainboard 130 in the machine head via Bluetooth, the display screen 111 displays the medicine compartment position, and the medicine compartment position is sent to the cloud platform at the same time, and is synchronously displayed on the mobile terminal APP; a partition board LED button light backboard is provided at a position on the back of the partition board 225 corresponding to the partition board LED button light 226 for controlling the partition board LED button light 226. The present disclosure can also use a high-sensitivity pressure sensor to automatically identify taking and putting medicines.

The manual control of the partition board LED button light 226 can be used in the following scenarios:
scenario 1: taking medicine on time. Take out the medicine from the designated compartment and put it back to the compartment with the LED light on after taking the medicine.
scenario 2: adding medicines. Scan the code to identify the medicines, then put them into the medicine box compartment, and press the button to return to the medicine storage compartment information.

In scenario 1, the steps are as follows:
1. Arrive at the preset medication time: The system is activated when the scheduled medication time arrives.
2. Voice broadcast of the medicine name and dosage, the screen displays the medicine name and dosage, and the medicine LED lights up: The system broadcasts the name and dosage of the medicine, and the corresponding LED light in the medicine cabinet lights up to remind the user to take the medication.
3. The user decides whether to press the function key to interrupt the broadcast or open the medicine cabinet:

If the user presses the function key, the system stops the voice broadcast, and the screen prompts the user to confirm whether the medication has been taken.

If the user does not press the function key, the system will issue a single reminder three times, with a 2-minute interval between each reminder, repeating a total of five times.
4. The user takes out the medicine from the indicated compartment according to the prompt and takes the medication as directed: The user needs to take the medicine from the compartment with the lit LED and follow the instructions to take the medication.
5. Close the medicine cabinet: After taking the medicine, the user closes the cabinet.
6. The user decides whether to press the function key to confirm that the medication is complete:

If the user presses the function key, the LED light in the medicine cabinet goes out, indicating that the medication has been taken.

If the user does not press the function key to confirm, the system will issue another reminder after 30 minutes. If the user still does not press the function key to confirm, the system will record the medication as missed for this instance.
7. The medicine cabinet and compartment lights go out, indicating that the medication is complete: Once the medication is confirmed as complete, the lights in the cabinet and the compartment turn off, signaling the end of the medication process.
8. Record the medication status and generate a medication report (viewable through the children's APP): The system records the user's medication status and generates a report, which can be viewed by the user or their children through the APP.
9. Complete the medication process: After confirming the medication is complete, the entire process is finished.

In scenario 2, the steps are as follows:
1. Scan the medicine barcode to query the medicine information: The user starts querying the medicine information by scanning the medicine barcode.
2. Is the medicine information found:

If no medicine information is found, the system will prompt via the screen or voice broadcast: “No medicine information found. If you need to set up a medication plan, please add it manually,” and ask the user to enter the medicine name.

If medicine information is found, the system will prompt via the screen or voice broadcast: “Medicine name, indications, please set up the medication plan.”
3. Select the medicine cabinet and compartment position:

The user opens the medicine cabinet and presses the LED button of the compartment where the medicine will be stored, or selects the medicine cabinet and compartment position via the screen or voice prompts.

The selection can be made through any terminal device, and the data is communicated via Bluetooth.
4. Screen/voice guidance to set up the medication plan: The system guides the user to set up the medication plan via the screen or voice prompts, including details such as the medication cycle, frequency, reminder time, and dosage.
5. Place the medicine into the designated compartment of the medicine cabinet: According to the system's instructions, the user places the medicine into the designated compartment of the medicine cabinet.
6. Complete the medication setup: Once the medicine is placed into the compartment and the medication plan is set, the process is complete.

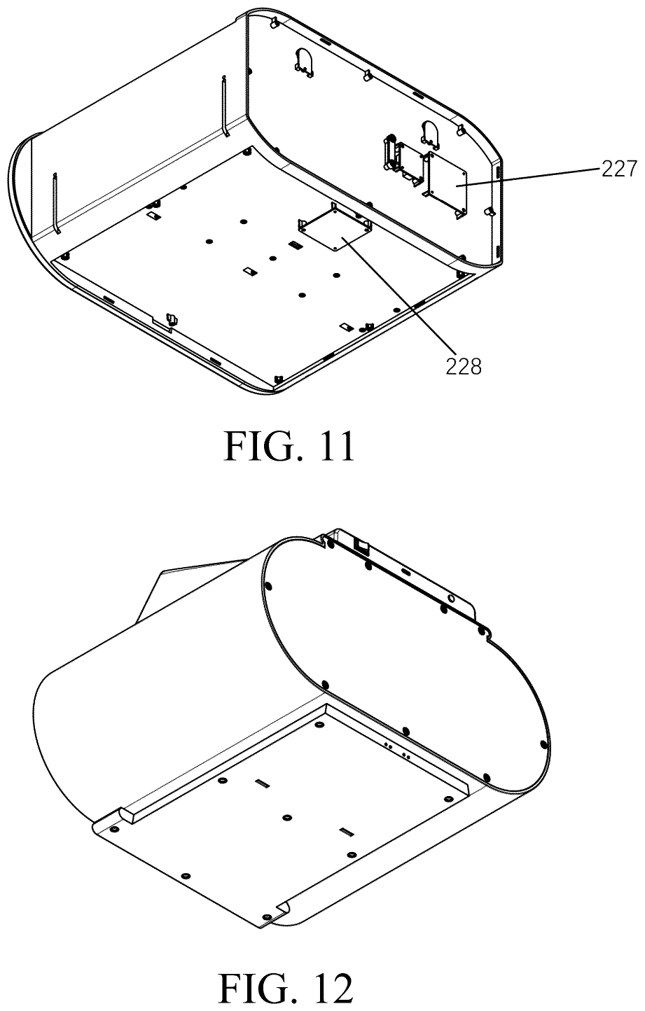

The medicine storage drawer 220 further comprises: a Hall sensor board 227 and a Bluetooth main control board 228; the Hall sensor board 227 is installed at the rear of the medicine storage drawer body, and the Bluetooth main control board 228 is installed at the bottom of the medicine storage drawer body, as shown in FIG. 11;
the Hall sensor board 227 is electrically connected to the Bluetooth main control board 228 at the bottom of the medicine storage drawer 220, as shown in FIG. 14; the Hall sensor board 227 corresponds to the position of the Hall sensor magnet 216 provided on the box shell 210, and senses the opening and closing state of the medicine storage drawer 220 through the contact between the Hall sensor board 227 and the Hall sensor magnet 216, and transmits the state signal to the Bluetooth main control board 228 through the wire;
the Bluetooth main control board 228 is also electrically connected to the back panel of the partition board LED button light, used to supply power to the partition board LED button light 226. The LED light status signal and button status signal are sent to the Bluetooth control board through wires, the Bluetooth control board sends the medicine grid position signal to the head Bluetooth antenna array board 160 through Bluetooth, and the Bluetooth antenna array board 160 sends the signal to the main control board through wires. The main control board sends the position signal to the cloud through WIFI.

The Bluetooth main control board 228 receives the status signal of the partition board LED button light 226 and the opening and closing status signal of the medicine box by the Hall sensor, and sends the signal to the Bluetooth antenna array board 160 and/or the Bluetooth module of the machine head, and then the Bluetooth antenna array board 160 sends the signal to the control mainboard 130 of the machine head 100; the Bluetooth main control board 228 supplies power to the partition board LED button light 226 and the Hall sensor board 227.

Preferably, the Bluetooth main control board 228 is located at the bottom of the medicine box drawer, directly below the Bluetooth antenna array board 160 in the machine head 100, to achieve the best Bluetooth communication effect.

The box bottom pin 214 is arranged at the lower part of the rear part of the box shell 210; the power distribution board includes three circuit interfaces, one of which inputs a power supply signal from the power supply pin 213 at the top of the medicine storage box 200, and the other two are respectively connected to the pins at the bottom of the box through wires, and connected to a Bluetooth main control board 228 on the medicine storage drawer 220 through a spring power line, respectively supplying power to the medicine storage box 200 and the Bluetooth main control board 228; the Hall sensor magnet 216 corresponds to the position of the Hall sensor board 227 arranged on the medicine storage drawer 220;
the scalable structure of the spring power line ensures that power can still be supplied when pushing or pulling the drawer.

When there is more than one medicine storage box 200 in the multifunctional intelligent medicine box, different medicine storage boxes are connected through a T-shaped structural base set at the bottom of the outer shell of the medicine storage box 200. The box mounting buckle set at the bottom of the T-shaped structural base is matched with the U-shaped slide rail structure 211 and the mounting slot on the top of the adjacent medicine storage box 200 to connect and fix; the Bluetooth control panels of different medicine storage drawers can communicate with the Bluetooth antenna array board 160 on the machine head to realize the positioning and automatic numbering of multiple medicine storage boxes 200, and multiple medicine storage boxes can also be manually numbered.

The multifunctional intelligent medicine box of the present disclosure can store medicines whether packaged or not because the space divided in the medicine storage drawer is relatively large.

The intelligent medicine box with an expansion structure in the present disclosure is a desktop type intelligent medicine box that can set, remind, and manage the user's medication; the intelligent medicine box with an expansion structure in the present disclosure can realize the functions of storing medicines, voice playback and setting, screen display and input, remote child care, medicine storage box superposition expansion, internal positioning of medicines, automatic acquisition of medicine information, constant temperature storage of medicines, etc.

Specifically, the intelligent medicine box with an extended structure in the present disclosure can directly put packaged medicines; voice reminders for taking medicines at regular intervals and in fixed quantities, and voice input to set medications; the screen displays equipment and medication information, and the screen input to set medications; medications are set through WIFI wireless network, and medication status is fed back to realize the remote care function of children's APP; the medicine storage box can be stacked and expanded to realize the function of expanding the storage space for medicines, and multiple stacked medicine box boxes can be automatically numbered manually or based on the Bluetooth cm-level positioning function; Bluetooth communication is combined with the medicine grid LED button to achieve accurate positioning of medicines in the medicine box, and the medicine position is synchronized to the smart display terminal; during the process of putting in and extracting medicines, the medicine grid LED button is pressed, the medicine grid LED light lights up, and the screen synchronously displays the corresponding position; the medicine grid position signal is sent to the head smart display terminal through Bluetooth communication; the camera scans the barcode on the medicine packaging, obtains medicine information, and synchronizes voice broadcast and screen display; constant temperature medicine storage, etc.

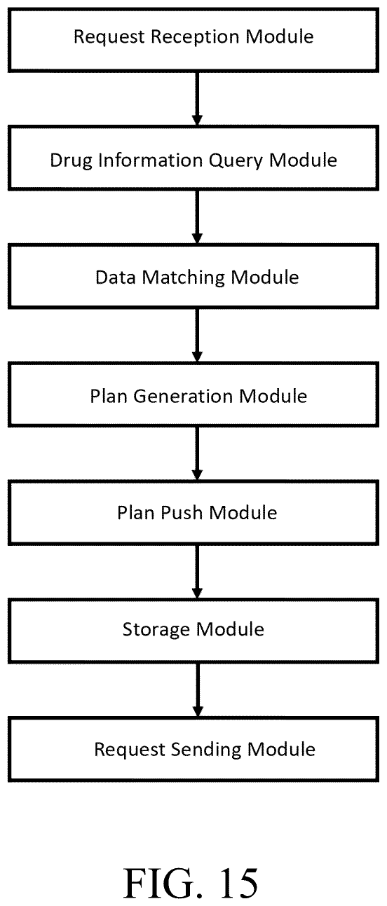

A medication reminder system based on a multifunctional intelligent medicine box, as shown in FIG. 15, includes:
a request receiving module, used to receive the medicine placing, taking and taking data sent by the smart medicine box;
a medicine information query module, used to query medicine information;
a data matching module, used for matching the user's medication plan according to the medicine information;
a plan generation module, used to generate a medication plan when there is no matching medication plan for the medicine information;
a plan push module, used to send the medication plan reminder to the user terminal, and the medication plan reminder is used to instruct the intelligent medicine box to remind the user to take the medicine;
a storage module, used for storing medicine information and medication plans; and
a request sending module, used to send the medication plan and medication records to the cloud server, and the child-side application can remotely view the medication records.

### Embodiment 1

This embodiment provides an intelligent medicine box with an extended structure, the intelligent medicine box comprising a machine head 100, a medicine storage box 200;
the machine head 100 includes a machine head body 110 and a machine head base 120, and the machine head body 110 and the machine head base 120 are an integrated structure;
the medicine storage box 200 comprises: a box shell 210 and a medicine storage drawer 220, wherein the medicine storage drawer 220 can be pulled out and moved inside the box shell 210;
the machine head 100 matches the top structure of the medicine storage box 200, so that the machine head 100 can be fixedly connected to the medicine storage box 200.

The machine head base 120 is a T-shaped structure, which matches the U-shaped slide rail structure protruding from the top of the medicine storage box 200, and the machine head base 120 is inserted into the U-shaped slide rail structure 211 at the top of the medicine storage box 200 through the positioning buckle at the bottom of the machine head base 120 and the positioning slot at the top of the medicine storage box 200; the front end of the U-shaped slide rail structure 211 is equipped with a tilt angle structure, which facilitates the stacking and fixation of the machine head 100 and multiple medicine boxes 200.

The two sides of the T-shaped structure of the machine head base 120 can slide and snap into the inner side of the slide rail of the U-shaped slide rail structure 211 at the top of the medicine storage box 200; the tail of the machine head base 120 is a step-shaped structure, which matches and snaps with the bottom shape of the U-shaped slide rail structure 211 at the top of the medicine storage box 200; the positioning buckle 121 at the bottom of the machine head base 120 corresponds to the position of the positioning slot 212 at the top of the medicine storage box 200, and when the machine head 100 is fixedly connected to the medicine storage box 200, the positioning buckle 121 snaps into the positioning slot 212;
the step-shaped structure of the machine head base 120 is provided with a pin interface 122 which contacts a power supply pin 213 at the bottom of the U-shaped slide rail structure 211 at the top of the medicine storage box 200 to supply power to the medicine storage box 200; the pin interface 122 of the machine head is connected to a power control board 140 via a wire.

The front of the head body 110 is provided with a display screen 111, a scanning camera 112, function buttons 113, and a volume adjustment button;
the volume adjustment button comprises a volume up button 114 and a volume down button 115;
the scanning camera 112, the function buttons 113, and the volume adjustment buttons are distributed around the display screen 111;
a speaker hole 116 is arranged on the back of the machine head body 110.

A control mainboard 130 and the power control board 140 are fixedly arranged at the back of the display screen inside the machine head body 110; the control mainboard 130 comprises a voice module, a display module, and a WIFI module;
a lithium battery 150, a Bluetooth antenna array board 160, and a speaker cavity 170 are fixedly arranged at the bottom of the interior of the machine head body 110; the speaker cavity 170 is installed above the Bluetooth antenna array board 160 and corresponds to the position of the speaker hole 116 on the back of the machine head body 110;
the control mainboard 130 can also be equipped with a backup Bluetooth module, which can be used in case the Bluetooth antenna array board 160 fails for various reasons or in subsequent multi-channel signal upgrades.

The control mainboard 130, the power control board 140, the lithium battery 150, the Bluetooth antenna array board 160, and the speaker cavity 170 are electrically connected, and the processed signals transmitted between them are aggregated to the control mainboard 130;
the voice module is used to recognize and generate sounds; the display module is used to display the status information of the medicine box equipment, medication-related information and input medication information; the WIFI module is used to transmit device information and medication management information to the cloud platform; the power control board 140 is used as an electrical connection transfer structure to monitor the battery power, power connection status, and machine switch status;
the lithium battery 150 supplies power to the machine head 100 and/or medicine storage box 200; the Bluetooth antenna array board 160 is used to transmit Bluetooth communication signals and achieve Bluetooth communication between the machine head 100 and the medicine storage box 200; the speaker cavity 170 is used for external amplification of voice signals.

The lower side of the back of the machine head body 110 is also provided with a power switch 117, a power interface 118, and a network distribution button 119;
the power switch 117 is electrically connected to the power control board 140, used to control the start and shut down of the machine head 100;
the power interface 118 is electrically connected to the power control board 140 and is used to connect external wires to directly power the machine head 100 and/or charge the lithium battery 150 in the machine head 100;
the network distribution button 119 is electrically connected to the control mainboard 130 and is used to connect to the WIFI.

The rear portion of the box shell 210 is provided with a box bottom pin 214, a power distribution board 215, and a Hall sensor magnet 216;
the box bottom pin 214 is arranged at the lower part of the rear part of the box shell 210; the power distribution board includes three circuit interfaces, one of which inputs a power supply signal from the power supply pin 213 at the top of the medicine storage box 200, and the other two are respectively connected to the pins at the bottom of the box through wires, and connected to a Bluetooth main control board 228 on the medicine storage drawer 220 through a spring power line, respectively supplying power to the medicine storage box 200 and the Bluetooth main control board 228; the Hall sensor magnet 216 corresponds to the position of the Hall sensor board 227 arranged on the medicine storage drawer 220.

The medicine storage drawer 220 comprises: a medicine storage drawer body; the medicine storage drawer body comprises: a front panel 222 and a drawer body 221; the front panel 222 is installed at the front end of the drawer body 221;
the front panel 222 is provided with a drawer handle 223, and the side surface of the drawer body is provided with a sliding groove; the sliding groove corresponds to a box slide rail provided inside the box shell 210;
a medicine box status LED light 224 is provided at the top of the front panel 222, and the medicine box status LED light 224 can display different colors according to different scenes. When there are multiple layers of medicine boxes, the medicine box status LED light 224 lights up to indicate the corresponding medicine box storing the medicine; for example, when it is time to take medicine, the LED light on the front panel 222 of the medicine box storing the medicine in the multiple layers of medicine boxes will light up, helping users to accurately find the location of the medicine box where the medicine is stored.

Inside the medicine storage drawer body is divided into medicine compartments of different sizes by pluggable partition boards 225; a protrusion is provided on the upper part of the partition board of each medicine compartment, and a partition board LED button light 226 is provided at the position of the protrusion; after putting in and taking out medicines, the partition board LED button light 226 can be manually pressed to indicate the position of medicine; a partition board LED button light backboard is provided at a position on the back of the partition board 225 corresponding to the partition board LED button light 226 for controlling the partition board LED button light 226.

The medicine storage drawer 220 further comprises: a Hall sensor board 227 and a Bluetooth main control board 228; the Hall sensor board 227 is installed at the rear of the medicine storage drawer body, and the Bluetooth main control board 228 is installed at the bottom of the medicine storage drawer body;
the Hall sensor board 227 is electrically connected to the Bluetooth main control board 228 at the bottom of the medicine storage drawer 220; the Hall sensor board 227 corresponds to the position of the Hall sensor magnet 216 provided on the box shell 210, and senses the opening and closing state of the medicine storage drawer 220 through the contact between the Hall sensor board 227 and the Hall sensor magnet 216, and transmits the state signal to the Bluetooth main control board 228 through the wire.

The Bluetooth main control board 228 receives the status signal of the partition board LED button light 226 and the opening and closing status signal of the medicine box by the Hall sensor, and sends the signal to the Bluetooth antenna array board 160 and/or the Bluetooth module of the machine head, and then the Bluetooth antenna array board 160 sends the signal to the control mainboard 130 of the machine head 100; the Bluetooth main control board 228 supplies power to the partition board LED button light 226 and the Hall sensor board 227.

When using the intelligent medicine box with an extended structure in this embodiment for the medication process, the intelligent medicine box in the present disclosure can perform timed, quantitative, positioning voice broadcasting and screen display. Specifically, at the preset medication time, the voice broadcasts the medicine name and dosage, and the screen automatically lights up to display the medicine name and dosage information. The LED light at the front of the medicine box drawer lights up to remind you to open the drawer and take the medicine. After opening the medicine box, the corresponding LED light of the medicine compartment lights up. Take out the medicine and put it back in after taking it. Press the LED button to close the medicine box. The LED lights of the medicine compartment and medicine box turn off, indicating that the medicine has been taken on time. The signal is sent to the cloud platform to record the medication situation and generate a medication report; The children's application of the intelligent medicine box can remotely view the medication records of the user.

During the medication process, if you want to stop the voice broadcast, you can long press the function button below the machine head screen to stop the voice broadcast.

If the user fails to take the medication on time, such as not opening the drawer of the medicine box during the medication time, the medicine box will repeatedly remind the user to take the medication through voice prompts 3 times. After a 2-minute interval, the reminder will be repeated 3 times, repeating this process 5 times before stopping the broadcast. If no timely medication signal is received after 30 minutes, the cloud platform will issue a warning message to the children's application. Initiate human intervention measures.

### Embodiment 2

This embodiment provides an intelligent medicine box with an extended structure, the intelligent medicine box comprising a machine head 100, two medicine storage boxes 200;
the machine head 100 includes a machine head body 110 and a machine head base 120, and the machine head body 110 and the machine head base 120 are an integrated structure;
the medicine storage box 200 comprises: a box shell 210 and a medicine storage drawer 220, wherein the medicine storage drawer 220 can be pulled out and moved inside the box shell 210;
the machine head 100 matches the top structure of the medicine storage box 200, so that the machine head 100 can be fixedly connected to the medicine storage box 200.

The machine head base 120 is a T-shaped structure, which matches the U-shaped slide rail structure protruding from the top of the medicine storage box 200, and the machine head base 120 is inserted into the U-shaped slide rail structure 211 at the top of the medicine storage box 200 through the positioning buckle at the bottom of the machine head base 120 and the positioning slot at the top of the medicine storage box 200; the front end of the U-shaped slide rail structure 211 is equipped with a tilt angle structure, which facilitates the stacking and fixation of the machine head 100 and multiple medicine boxes 200.

The two sides of the T-shaped structure of the machine head base 120 can slide and snap into the inner side of the slide rail of the U-shaped slide rail structure 211 at the top of the medicine storage box 200; the tail of the machine head base 120 is a step-shaped structure, which matches and snaps with the bottom shape of the U-shaped slide rail structure 211 at the top of the medicine storage box 200; the positioning buckle 121 at the bottom of the machine head base 120 corresponds to the position of the positioning slot 212 at the top of the medicine storage box 200, and when the machine head 100 is fixedly connected to the medicine storage box 200, the positioning buckle 121 snaps into the positioning slot 212;
the step-shaped structure of the machine head base 120 is provided with a pin interface 122 which contacts a power supply pin 213 at the bottom of the U-shaped slide rail structure 211 at the top of the medicine storage box 200 to supply power to the medicine storage box 200; the pin interface 122 of the machine head is connected to a power control board 140 via a wire.

The front of the head body 110 is provided with a display screen 111, a scanning camera 112, function buttons 113, and a volume adjustment button;
the volume adjustment button comprises a volume up button 114 and a volume down button 115;
the scanning camera 112, the function buttons 113, and the volume adjustment buttons are distributed around the display screen 111;
a speaker hole 116 is arranged on the back of the machine head body 110.

A control mainboard 130 and the power control board 140 are fixedly arranged at the back of the display screen inside the machine head body 110; the control mainboard 130 comprises a voice module, a display module, and a WIFI module;
a lithium battery 150, a Bluetooth antenna array board 160, and a speaker cavity 170 are fixedly arranged at the bottom of the interior of the machine head body 110; the speaker cavity 170 is installed above the Bluetooth antenna array board 160 and corresponds to the position of the speaker hole 116 on the back of the machine head body 110;
the control mainboard 130 can also be equipped with a backup Bluetooth module, which can be used in case the Bluetooth antenna array board 160 fails for various reasons or in subsequent multi-channel signal upgrades.

The control mainboard 130, the power control board 140, the lithium battery 150, the Bluetooth antenna array board 160, and the speaker cavity 170 are electrically connected, and the processed signals transmitted between them are aggregated to the control mainboard 130;
the voice module is used to recognize and generate sounds; the display module is used to display the status information of the medicine box equipment, medication-related information and input medication information; the WIFI module is used to transmit device information and medication management information to the cloud platform; the power control board 140 is used as an electrical connection transfer structure to monitor the battery power, power connection status, and machine switch status;
the lithium battery 150 supplies power to the machine head 100 and/or medicine storage box 200; the Bluetooth antenna array board 160 is used to transmit Bluetooth communication signals and achieve Bluetooth communication between the machine head 100 and the medicine storage box 200; the speaker cavity 170 is used for external amplification of voice signals.

The lower side of the back of the machine head body 110 is also provided with a power switch 117, a power interface 118, and a network distribution button 119;
the power switch 117 is electrically connected to the power control board 140, used to control the start and shut down of the machine head 100;
the power interface 118 is electrically connected to the power control board 140 and is used to connect external wires to directly power the machine head 100 and/or charge the lithium battery 150 in the machine head 100;
the network distribution button 119 is electrically connected to the control mainboard 130 and is used to connect to the WIFI.

A U-shaped slide rail structure 211 and a positioning slot 212 are arranged on the top of the shell of the medicine storage box 200, and a power supply pin 213 is arranged at the bottom of the U-shaped slide rail structure 211 on the top of the medicine storage box 200; a T-shaped structure base is arranged at the bottom of the shell of the medicine storage box 200, and a box installation buckle is arranged at the bottom of the T-shaped structure base, and a box bottom pin 214 is arranged at the rear of the T-shaped structure base; the T-shaped structure base corresponds to the U-shaped slide rail structure 211, and the box bottom pin 214 contacts with the power supply pin 213 at the bottom of the U-shaped slide rail structure 211 on the top of other medicine storage boxes 200 to supply power to other medicine storage boxes 200.

In this embodiment, by combining the T-shaped structure base at the bottom of the previous box with the U-shaped slide rail structure 211 at the top of the next box, the number of storage boxes in the intelligent medicine box can be expanded.

The rear portion of the box shell 210 is provided with a box bottom pin 214, a power distribution board 215, and a Hall sensor magnet 216;
the box bottom pin 214 is arranged at the lower part of the rear part of the box shell 210; the power distribution board includes three circuit interfaces, one of which inputs a power supply signal from the power supply pin 213 at the top of the medicine storage box 200, and the other two are respectively connected to the pins at the bottom of the box through wires, and connected to a Bluetooth main control board 228 on the medicine storage drawer 220 through a spring power line, respectively supplying power to the medicine storage box 200 and the Bluetooth main control board 228; the Hall sensor magnet 216 corresponds to the position of the Hall sensor board 227 arranged on the medicine storage drawer 220.

The medicine storage drawer 220 comprises: a medicine storage drawer body; the medicine storage drawer body comprises: a front panel 222 and a drawer body 221; the front panel 222 is installed at the front end of the drawer body 221;
the front panel 222 is provided with a drawer handle 223, and the side surface of the drawer body is provided with a sliding groove; the sliding groove corresponds to a box slide rail provided inside the box shell 210;
a medicine box status LED light 224 is provided at the top of the front panel 222, and the medicine box status LED light 224 can display different colors according to different scenes. When there are multiple layers of medicine boxes, the medicine box status LED light 224 lights up to indicate the corresponding medicine box storing the medicine; for example, when it is time to take medicine, the LED light on the front panel 222 of the medicine box storing the medicine in the multiple layers of medicine boxes will light up, helping users to accurately find the location of the medicine box where the medicine is stored.

Inside the medicine storage drawer body is divided into medicine compartments of different sizes by pluggable partition boards 225; a protrusion is provided on the upper part of the partition board of each medicine compartment, and a partition board LED button light 226 is provided at the position of the protrusion; after putting in and taking out medicines, the partition board LED button light 226 can be manually pressed to indicate the position of medicine; a partition board LED button light backboard is provided at a position on the back of the partition board 225 corresponding to the partition board LED button light 226 for controlling the partition board LED button light 226.

The medicine storage drawer 220 further comprises: a Hall sensor board 227 and a Bluetooth main control board 228; the Hall sensor board 227 is installed at the rear of the medicine storage drawer body, and the Bluetooth main control board 228 is installed at the bottom of the medicine storage drawer body;
the Hall sensor board 227 is electrically connected to the Bluetooth main control board 228 at the bottom of the medicine storage drawer 220; the Hall sensor board 227 corresponds to the position of the Hall sensor magnet 216 provided on the box shell 210, and senses the opening and closing state of the medicine storage drawer 220 through the contact between the Hall sensor board 227 and the Hall sensor magnet 216, and transmits the state signal to the Bluetooth main control board 228 through the wire.

The Bluetooth main control board 228 receives the status signal of the partition board LED button light 226 and the opening and closing status signal of the medicine box by the Hall sensor, and sends the signal to the Bluetooth antenna array board 160 and/or the Bluetooth module of the machine head, and then the Bluetooth antenna array board 160 sends the signal to the control mainboard 130 of the machine head 100; the Bluetooth main control board 228 supplies power to the partition board LED button light 226 and the Hall sensor board 227.

When using the intelligent medicine box with an extended structure in this embodiment to query medicine information, the medicine barcode is mainly scanned by the scanning camera on the head to obtain the corresponding medicine information; specifically, long press the function button to activate the camera above the head screen, scan the barcode on the medicine package, and the intelligent medicine box transmits the medicine barcode information to the cloud platform via WIFI to query the medicine information. If the medicine information can be queried, it will check whether the medication plan has been set.

If the medicine information can be found and a medication plan has been set up, the cloud platform will send the medicine information to the medicine box and inform the patient of the medicine information and medication plan through voice broadcast and screen display.

If the medicine information can be queried, but there is no medication plan, the cloud platform will send the medicine information to the medicine box, which will voice broadcast and display the medicine information on the screen. It will then voice query and screen display to ask whether a medication plan needs to be set up. After receiving the user's voice and screen feedback, if a medication plan needs to be set up, the medication setting will be entered. If a medication plan does not need to be set up, the voice broadcast and screen display will be completed, and the user only needs to query the medicine information.

If no medicine information is found but a medication plan needs to be set up, the medicine name, dosage, and medication time can be entered through voice or screen input.

If the medicine information is not found and there is no need to set up a medication plan, the voice broadcast and screen display will indicate that the medicine information has not been found.

### Embodiment 3

This embodiment provides an intelligent medicine box with an extended structure, the intelligent medicine box comprising a machine head 100, and a plurality of medicine storage boxes 200.

When using the intelligent medicine box with an expanded structure in this embodiment to set the medication process, it can be set through voice or screen; specifically, if the medicine packaging is complete and there is a barcode on the packaging, first long press the function button below the screen of the medicine box head to activate the camera, scan the barcode information on the medicine packaging, and the medicine box will upload the barcode information to the cloud platform via WIFI. The cloud platform will match the information with the national medicine database to query whether the medicine barcode can be recognized and whether the medication plan for the medicine has been set up.

If the medicine information can be found and the medication plan has been set, the cloud platform will send the medicine information and medication plan to the medicine box. The medicine box will broadcast the medicine information by voice and display the medication plan on the screen. The LED indicator on the front panel of the medicine box drawer will light up. Pull open the medicine box, and the corresponding medicine compartment LED indicator will light up, and put the medicine in the designated medicine compartment with or without packaging. And press the medicine compartment LED button to indicate that the medicine has been put in.

If the medicine information cannot be found, the cloud platform will feed back the query results to the medicine box via WIFI, and voice broadcast and screen display will ask whether a medication plan needs to be set. When the reply needs to set medication, activate the voice input and screen manual input functions, enter the medicine name, medication time and dosage. The medicine box indicator light will light up, open the medicine box, select the medicine compartment to put the medicine in, press the medicine compartment LED button, and the medicine compartment information will be sent to the medicine box control terminal via Bluetooth. The medicine box will upload the medication information and medicine compartment location information to the cloud platform via WIFI.

If the medicine information can be found but there is no record of medication settings, it means that the medicine is entered for the first time. The cloud platform sends the medicine information back to the medicine box via WIFI, and the medicine box asks whether a medication plan needs to be set up through voice broadcast and screen display. When the reply is that medication needs to be set up, the system automatically enters the medicine name information and activates the voice entry and screen manual input functions. The user only needs to set the medication time and dosage. The other processes are as described above.

### Embodiment 4

The intelligent robot in this embodiment is a desktop type intelligent robot with functions such as companionship and interaction, daily life information query and broadcast, user health information query and broadcast, medication information reminder query and broadcast, and remote interactive calls with family members. Specific functions include:
1. Companionship interaction: scene+voice+expression+action, superimposed to generate interactive effects.

In addition to regular sports movements, there are also specific scene movements, such as the following two scenes.
1) Head shaking motion: There is one on the top of the robot's head and two on both sides of the cheeks, and patch capacitive touch sensors 32; Application scenario: When the user touches the robot's head and cheeks, the robot turns on the motors of its head and neck and shakes its head;
2) Emotional outbursts: The speed and amplitude of various movements of the body, head, and neck are amplified; Application scenarios: in scenarios where executive function fails, including speech recognition failure, etc.
2. Daily life information query and broadcast: Access the cloud platform via WIFI and broadcast daily life information based on user query needs.
3. Associated APP, user health information query and broadcast: Access the robot's exclusive cloud platform via WIFI, associate the robot's exclusive APP, and broadcast daily health status information based on user query needs.
4. Associated APP, user medication information query and broadcast: Access the robot's exclusive cloud platform via WIFI, associate the robot's exclusive APP, and regularly broadcast reminders to users to take medication; you can also query medication at any time and broadcast medication information based on user needs.

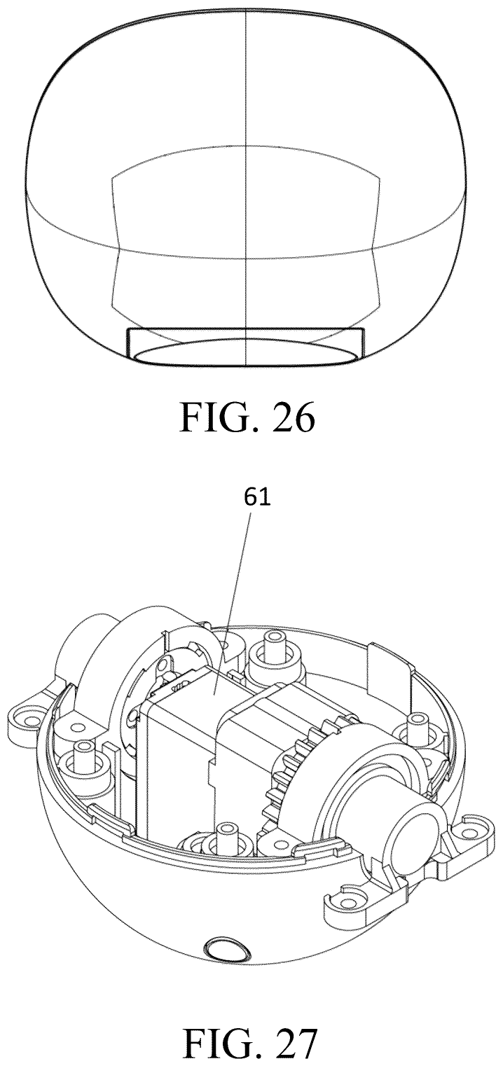

The intelligent robot in this embodiment has a structure including: a head mechanism A, a neck mechanism B, and a body mechanism C. The movement mode is that the head shakes left and right, the neck nods up and down, the body rotates, and the base remains stationary; the actions that can be realized include: wake-up action (the robot raises its head to a horizontal level, 90 degrees with the longitudinal axis), self-check action (the head moves left and right, the neck pitches, and the body rotates to the maximum), reset action (same as wake-up action), and cute action.
1. Head: control shaking motor, integrated display screen and frame, camera, transmission gear, dual microphones, capacitive touch sensor, main control board, shell;
as shown in FIG. 25 and FIG. 26, a side view and a rear view of the head mechanism are provided respectively.

The head shakes left and right with an amplitude of 80 degrees each, for a total of 160 degrees;
2. Neck: control nodding motor, gear structure, and housing;
the range of motion of the neck nodding up and down is 30 degrees up and down, for a total of 60 degrees;
3. Body: controls the body swing motor, sound cavity, sound outlet, and shell;
the body rotation range starts from 0 degrees and rotates in one direction up to 345 degrees. It is controlled by a limit switch;
4. Base: one power interface, multiple charging interfaces, network distribution button, machine switch button, rechargeable battery, power control board, motor control board, turntable base, and base frame.

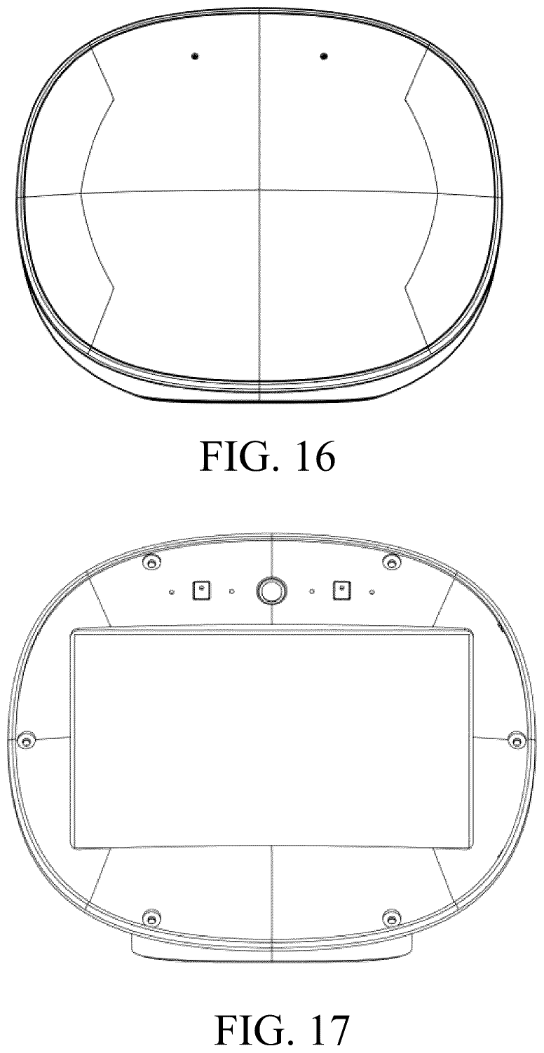

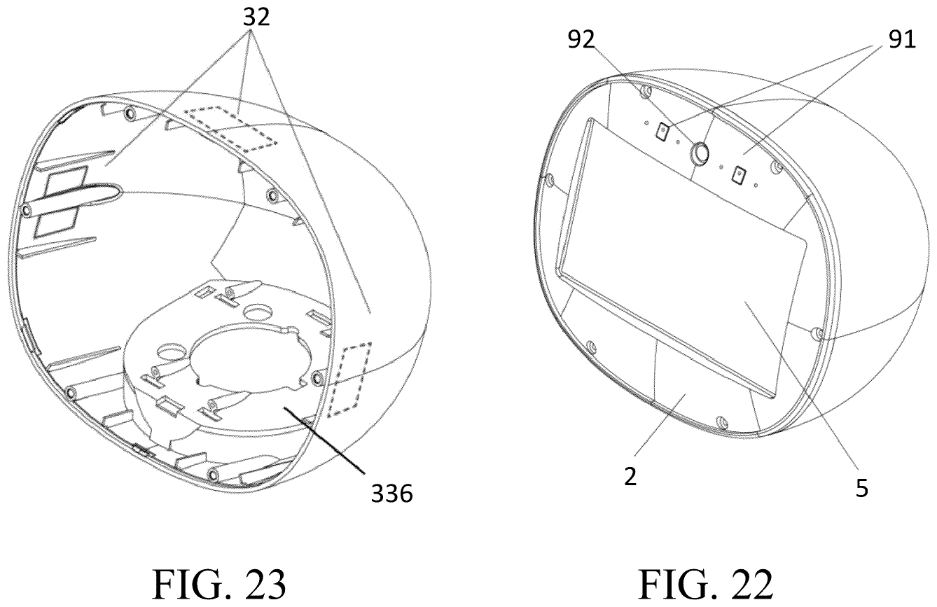

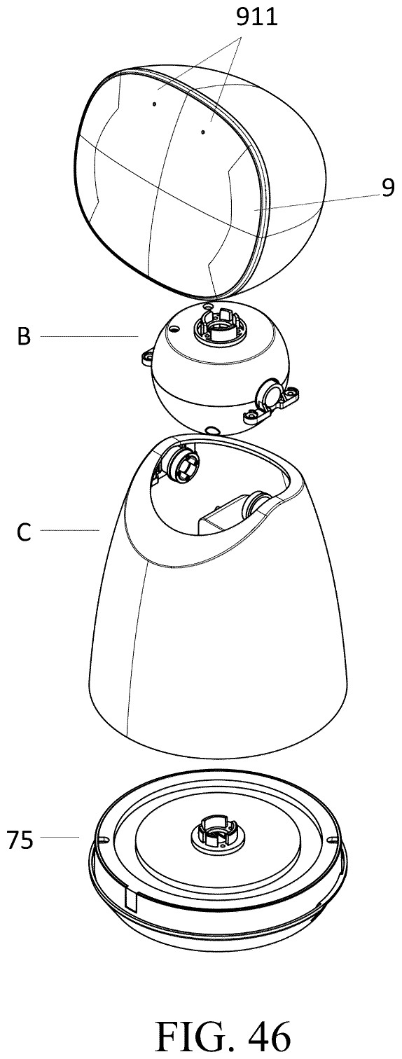

The intelligent robot in this embodiment comprises: a head mechanism A, a neck mechanism B, and a body mechanism C, as shown in FIG. 46;
the head mechanism comprises: a mask 9, a head bracket 10, a head body, a head shell 4, a display screen, a microphone 91, and a camera 92, which are all fixed on the head bracket 10 with screws, as shown in FIG. 22; the head mechanism with a mask or without a mask is shown in FIG. 16 and FIG. 17 respectively;
the head body comprises: a main control board 31, a capacitive touch sensor 32, a head DC motor and its transmission structure 33, as shown in FIG. 19; the main control board 31 is fixedly arranged at the back of the display screen inside the head body; the capacitive touch sensor 32 is glued and fixed in the head shell; the DC motor controls the robot's shaking movement and is fixed in the motor frame by screws, and the head motor frame 332 is fixed to the head shell 4 by screws, as shown in FIG. 21.

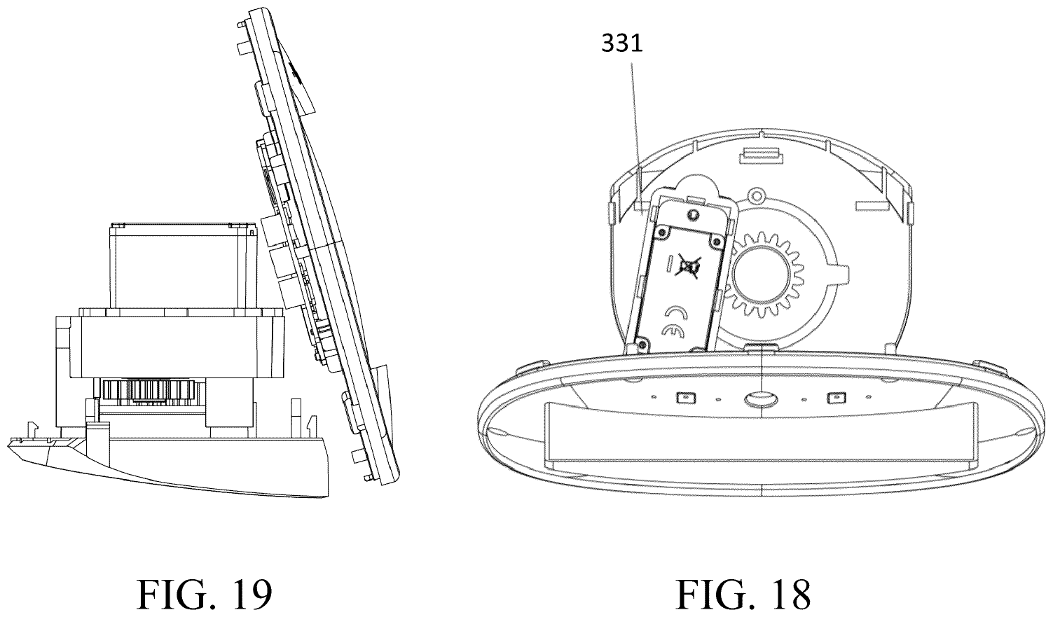

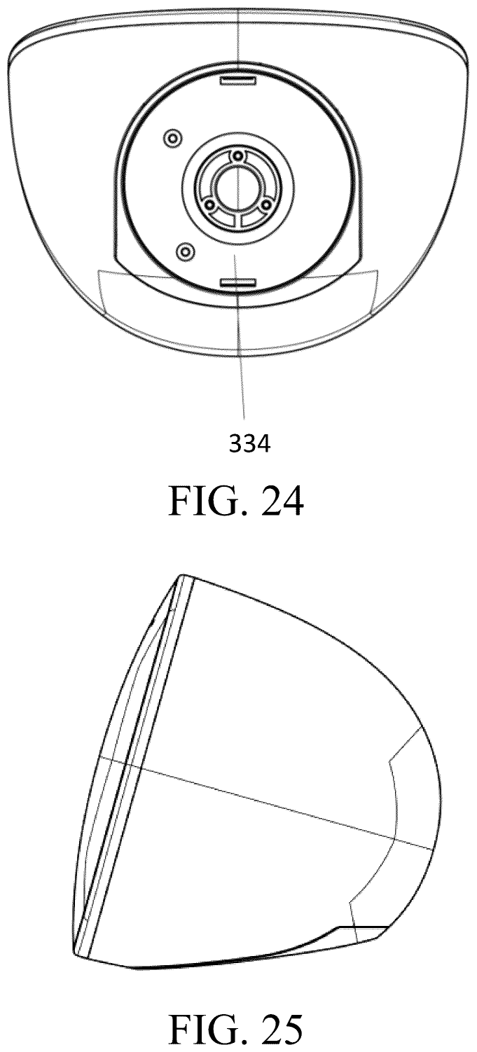

The head DC motor and its transmission structure comprise: head DC motor 331, head motor frame 332, head gear set 333, and head bearing 334, as shown in FIG. 18; head DC motor 331 drives head gear set 333 to rotate, head gear set 333 drives head bearing 334, head bearing 334 is fixed on the motor head shell 4, as shown in FIG. 24, and drives the head to shake left and right. With the position of the robot body LOGO as the center, the maximum rotation angle of the head to the left and right is 80 degrees.

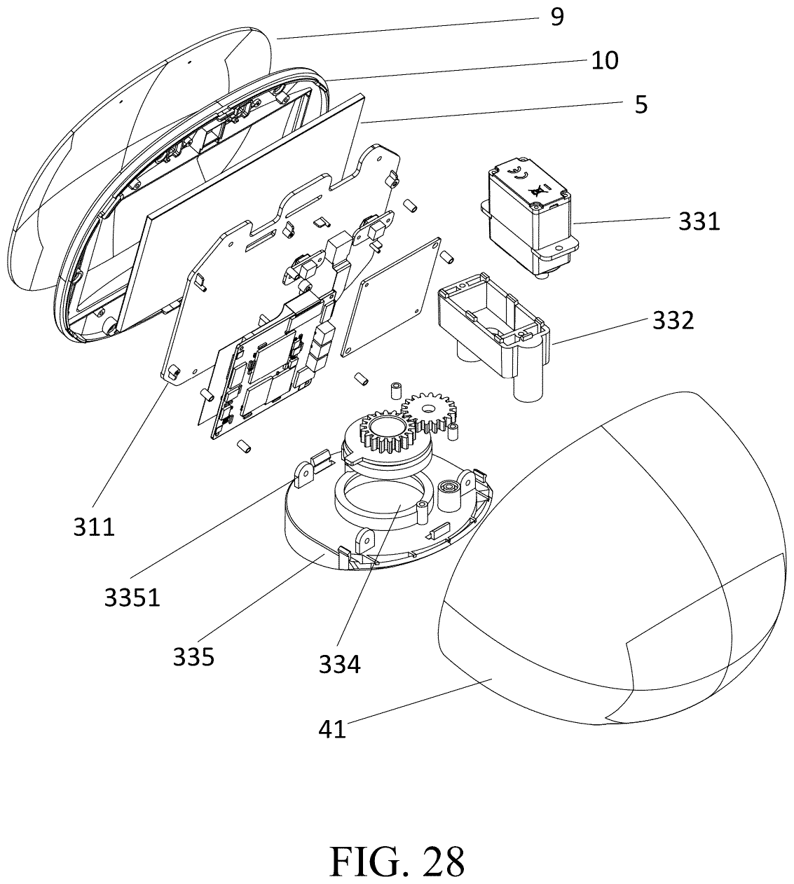

In the present disclosure, the head rear shell is provided with a positioning portion that is adapted to the shape of the head bottom shell and the head DC motor and its transmission structure; the fixed part on the bottom shell of the head is fixed to the positioning part 336 by screws through the screw fixing holes 3351, as shown in FIG. 23 and FIG. 28.

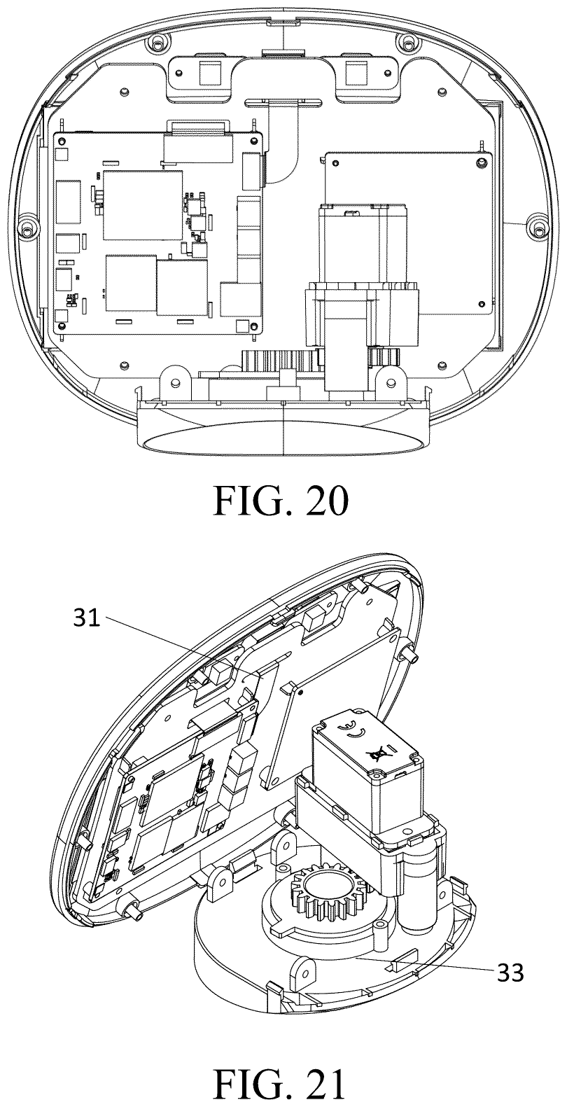

The capacitive touch sensor 32 senses when the user touches the outer shell, and communicates with the main control board 31 to transmit the touch sensing signal, as shown in FIG. 20. The main control board 31 controls the movement of the head, neck, and body motors through the motor control board. At the same time, the main control board locally starts the corresponding cute expressions and sounds, displays the cute expressions on the robot head display screen, and plays the preset cute sound effects through the speaker.

Specifically, in the head mechanism,
(1). Display screen:
a) Display device information: current time, battery level, charging mode, WIFI connection status, Bluetooth connection status.
b) Display realistic expressions.
c) Talk to family members remotely.
(2). Camera: Above the screen.
a) Track and locate the user's position. The robot always faces the user. When the user moves, the robot can track and locate the user's position through the camera.
b) Users can be filmed while talking to family members remotely.
c) Face recognition can recognize the user by face, and directly call the other party by nickname (or name) during voice communication.
d) Gesture recognition: set several commonly used gestures (such as waving twice or more to close the video; a one-handed OK gesture to indicate receipt of a medication reminder and interrupt the medication broadcast).
e) Dressing interaction: When users chat with the robot (after connecting to the voice model, the robot will give today's clothing suggestions based on the weather forecast and the user's current clothing), or praise the red robe worn today for being festive and the navy blue shirt for being tasteful.
(3). Dual microphones: on both sides of the camera.
a) Voice interaction. Voice signals do not pass through Bluetooth. Voice signals are received normally and processed by the voice module. Bluetooth function: 1. Network configuration; 2. Determine whether the medicine box and the robot are in the same room. When the robot and Bluetooth are in the same room, the medicine box broadcast is the main one.
b) User positioning: First, the approximate direction is determined by voice, and then the specific location is tracked by the camera.

Motor and gear: Control the robot's shaking motion. The motor's motion range is 80 degrees to the left and right, for a total of 160 degrees.
(4). Main control board: Behind the screen. It includes voice, display, WIFI, Bluetooth Low Energy (BLE) and other modules. The wires are connected to control the capacitive touch sensor, motor control board, power control board, and network distribution button.

The logic operation control of the motor is still completed on the main control board. The motor control board is the execution control of the motor movement.
(5). Bluetooth: used for network configuration.
(6). WIFI communication, access to cloud platform
a) Access the public cloud platform to query daily information: time, weather, temperature, playing songs, etc.
b) Access the dedicated cloud platform, link the APP, and query the user's daily health information: blood sugar, blood pressure, blood oxygen, etc.;
c) Access the dedicated cloud platform, link the APP, and query the user's daily medication information: medicine name, dosage, and time;
d) Access the dedicated cloud platform, link the APP, and query the user's home safety information: smoke alarms, water overflow alarms, security alarms and other information.
e) Talk to relatives remotely.
(7). Voice interaction: Realize voice recognition, analysis and synthesis on the cloud platform.
(8). Interaction: scene+voice+expression+action.

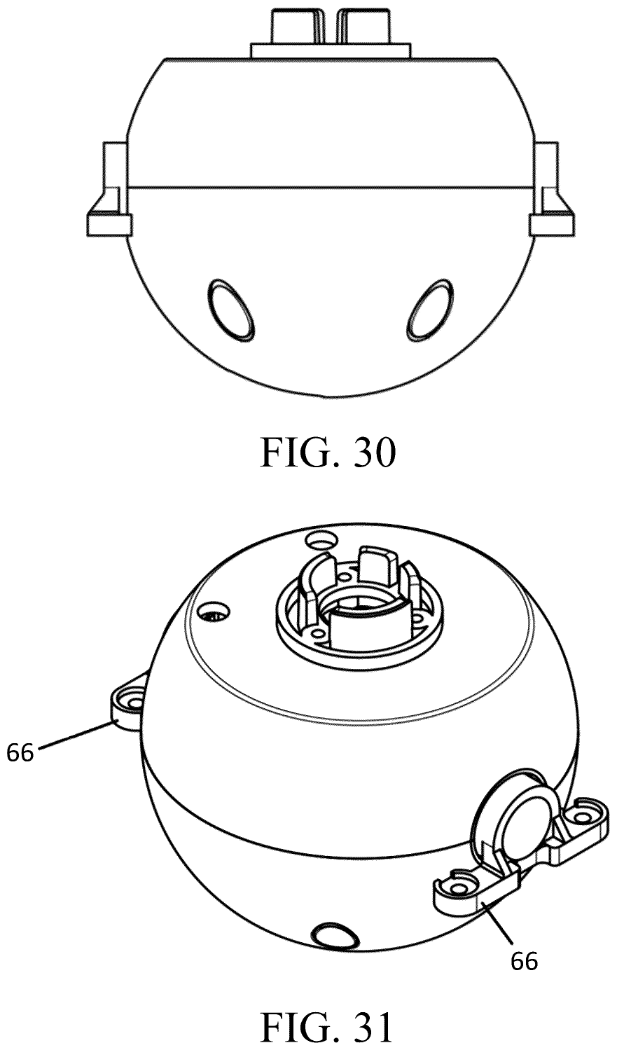

In specific scenarios, perform voice, expression, and action.
(9). Capacitive touch sensor
a) There is one on the top of the head and one on each side of the cheek, for a total of three.
b) Two scenarios:
scenario 1. Black screen, touch to wake up the robot;
scenario 2: The screen is not black and the device is not in sleep mode. The user touches the housing, triggering the head shaking action.

Wherein, the neck mechanism is fixedly connected to the bottom of the head by screws, and the two sides of the neck are fixedly connected to the body by screws. The neck connects the head and body, improves the load-bearing capacity, and increases the movement dimension and flexibility. As shown in FIG. 30, is a front view of the neck mechanism.

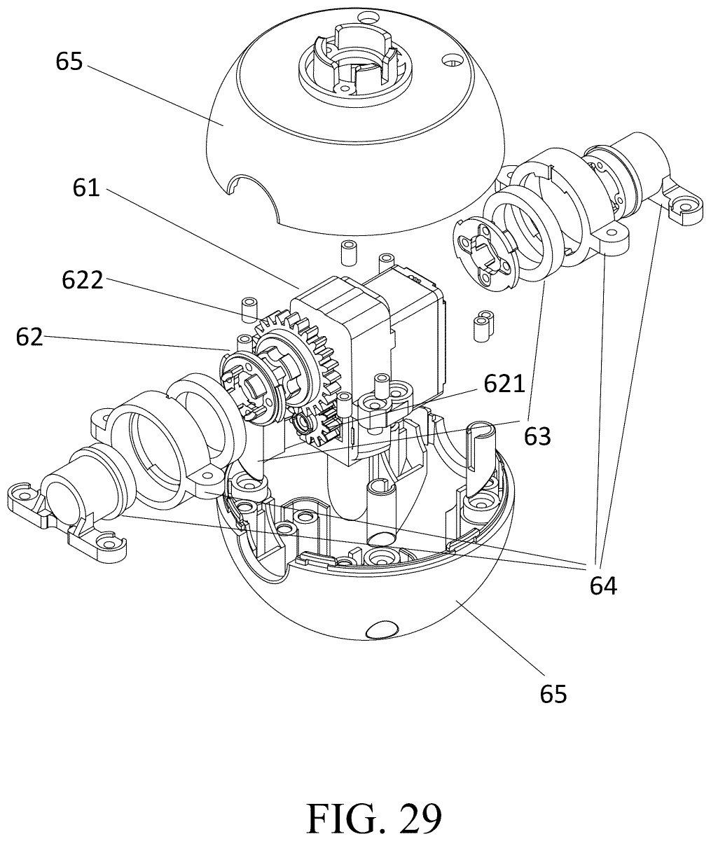

As shown in FIG. 27, the neck includes a neck DC motor 61, a neck gear set 62, double-sided bearings 63, a neck bracket 64 and upper and lower shells; the neck DC motor 61 controls the robot's nodding action through the neck gear set 62 and double-sided bearings 63; the motor shell is divided into an upper half and a lower half, and the upper and lower shells are connected and fixed by screws;
the neck movement is achieved by the neck DC motor 61 connected with the neck gear set 62 (including neck sun gear and neck planetary gear) and the bearing to realize the movement control, as shown in FIG. 29. The left and right side bearings are for movement balance.

The lower part of the upper and lower shells is designed in a semicircular style. The semicircular structure of the neck is an innovative structure, which has significant improvements in increasing dimensions, improving movement flexibility, and improving load-bearing capacity.

As shown in FIG. 31, the double-sided bearings on both sides of the neck mechanism are connected to the neck bracket, and the neck bracket is provided with a fixing part 66 that can be fixedly connected to the robot body mechanism.

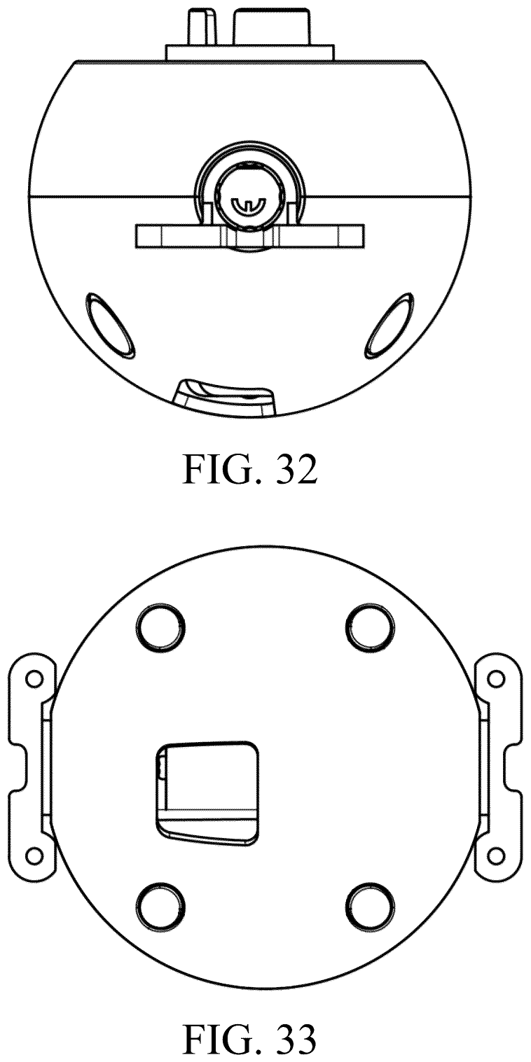

As shown in FIG. 32, a side view of the neck mechanism is provided.

As shown in FIG. 33, a bottom view of the neck mechanism is provided.

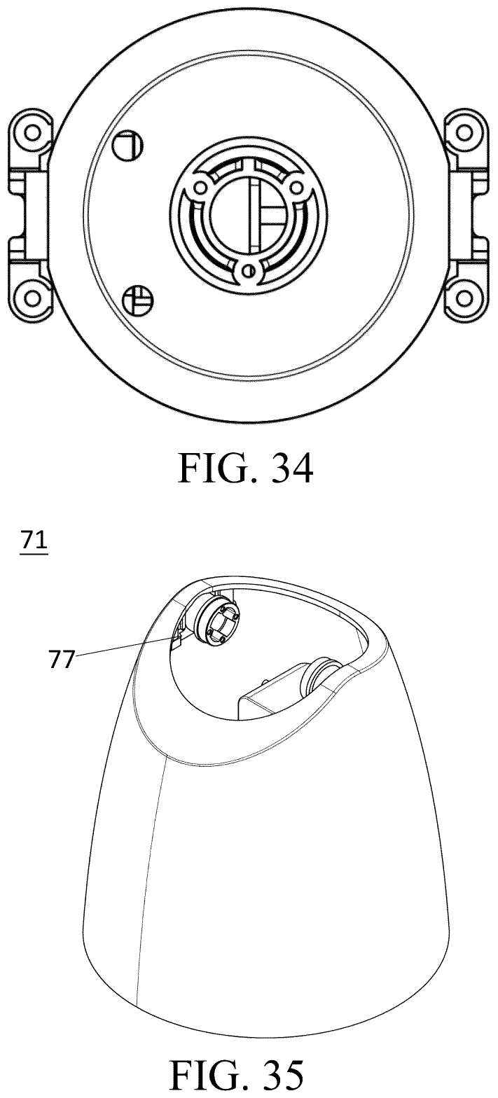

As shown in FIG. 34, a top view of the neck mechanism is provided.

There are screw holes inside the body mechanism, which correspond to the positions of the screw holes in the neck, and the neck and body are fixed by a screw connection.

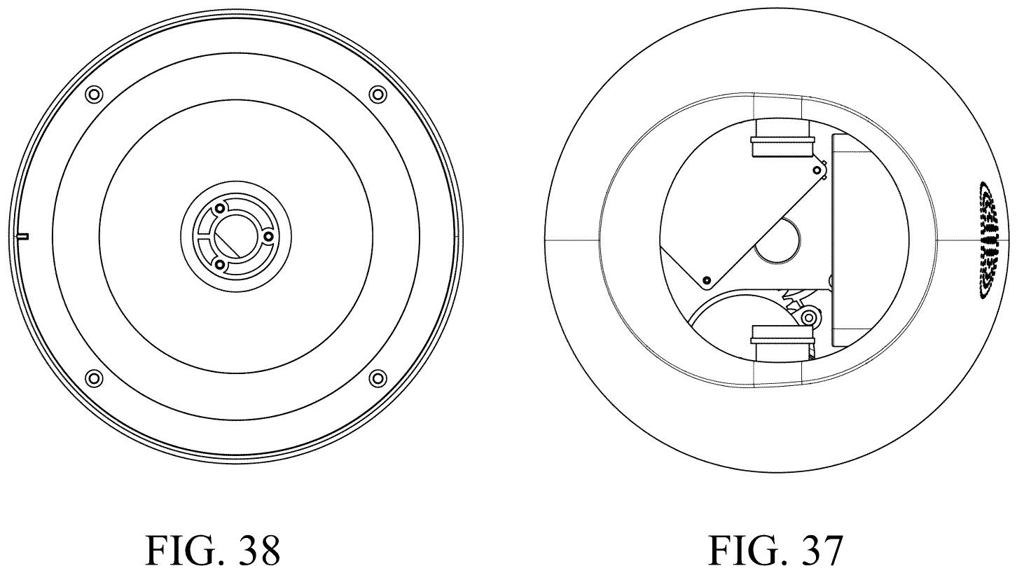

As shown in FIG. 37, a top view of the body mechanism is provided.

As shown in FIG. 38, a bottom view of the body mechanism is provided.

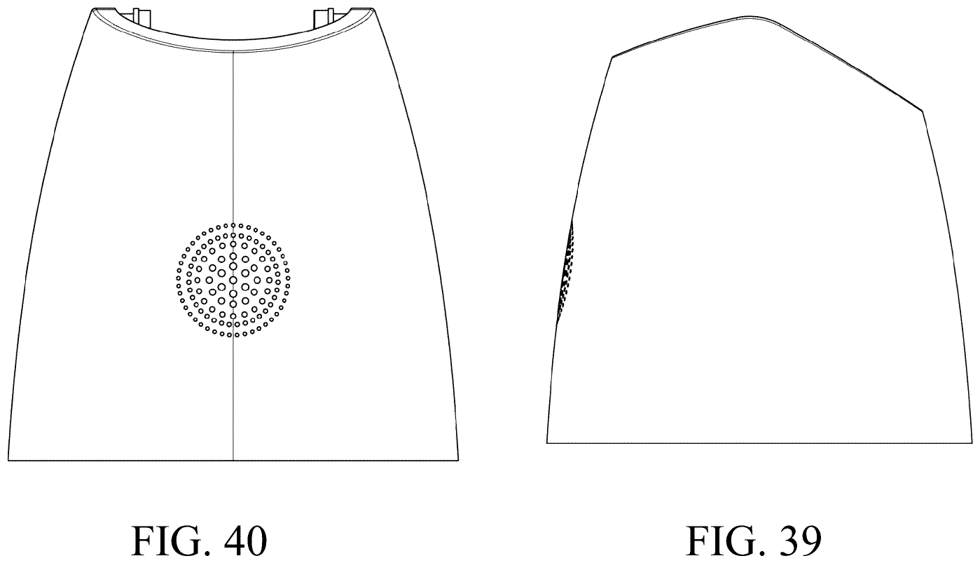

As shown in FIG. 39, a side view of the body mechanism is provided.

As shown in FIG. 40, a rear view of the body mechanism is provided.

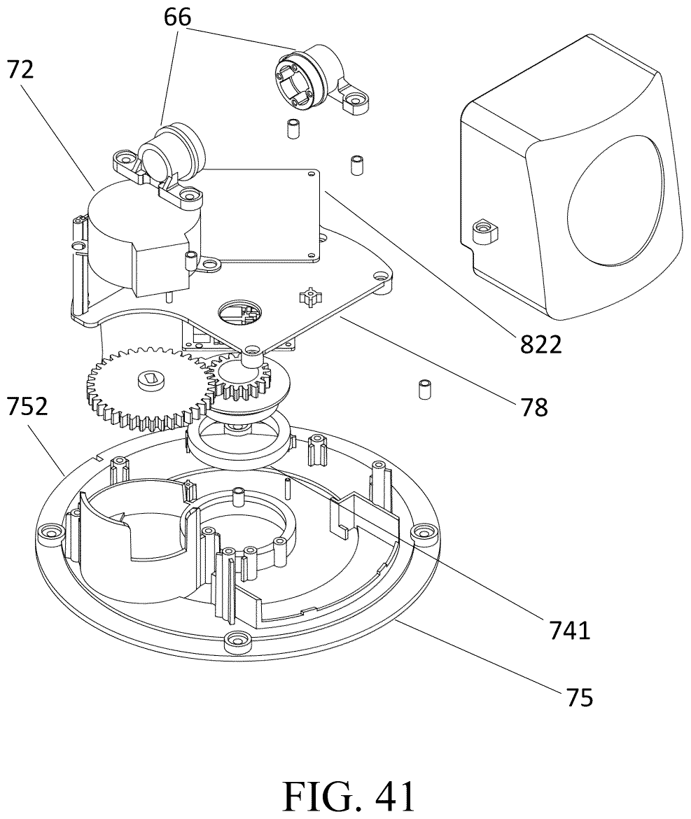

As shown in FIG. 41, a schematic diagram of the transmission structure of the turntable part of the body mechanism with neck shaft fixing frames is provided.

As shown in FIG. 43, a front view of the body mechanism is provided.

The body includes a speaker hole and a shell. A product logo is on the front of the robot body, which is used as the front base point; at the back position, there is a speaker hole 711, which corresponds to the position of the speaker cavity 73; the top of the shell is an arc shape, corresponding to the shape of the lower half of the neck shell, which is used to place the robot neck and facilitates the rotation of the robot neck;
the body is fixedly connected to the turntable base 751 by screws.

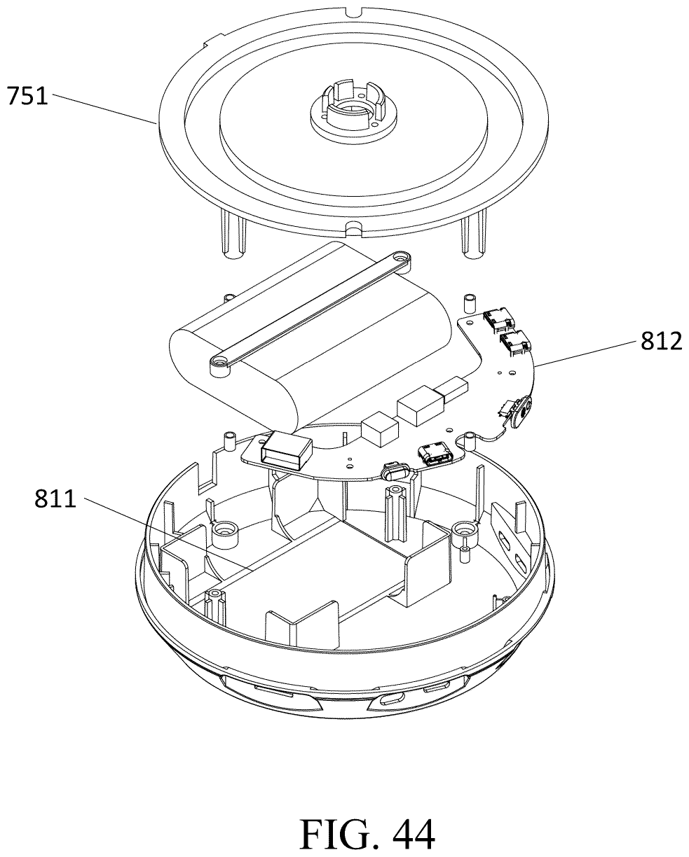

The base includes a base body 81 and two layers of a turntable body, and the turntable body is fixed to the base frame 811 by screws;
the base body includes a base frame 811, an input and output power interface, a power switch 816, a network distribution button 817, a rechargeable battery 813, and a power control board 812; the turntable body includes a turntable base 751, as shown in FIG. 44, a turntable DC motor and its transmission structure 72, a motor control board 822, and a speaker cavity 73.

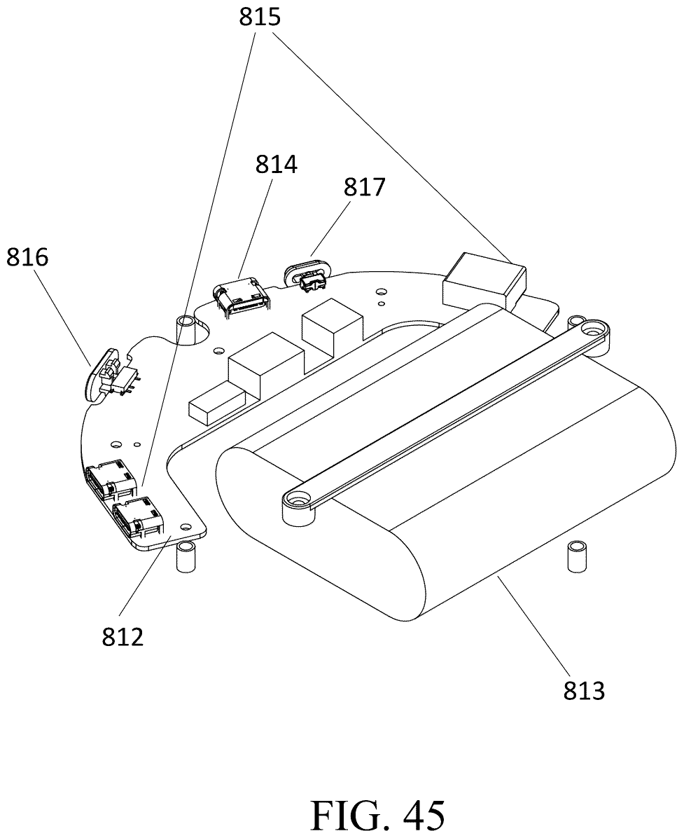

As shown in FIG. 45, the base frame includes a power input interface 814, multiple power output interfaces 815 (two Type C interfaces, one Type A interface), a network distribution button 817, a machine power switch button, a rechargeable battery 813, and a power control board 812. The input and output power interfaces, the machine power switch, and the rechargeable battery 813 are all connected to the power control board 812 through wires, and the power control board 812 is connected to the main control board of the robot head through wires. The network distribution button 817 is electrically connected to the main control board of the head.

The turntable body includes a turntable base 751, a turntable DC motor and its transmission structure 72, a motor control board 822, and a speaker cavity 73.

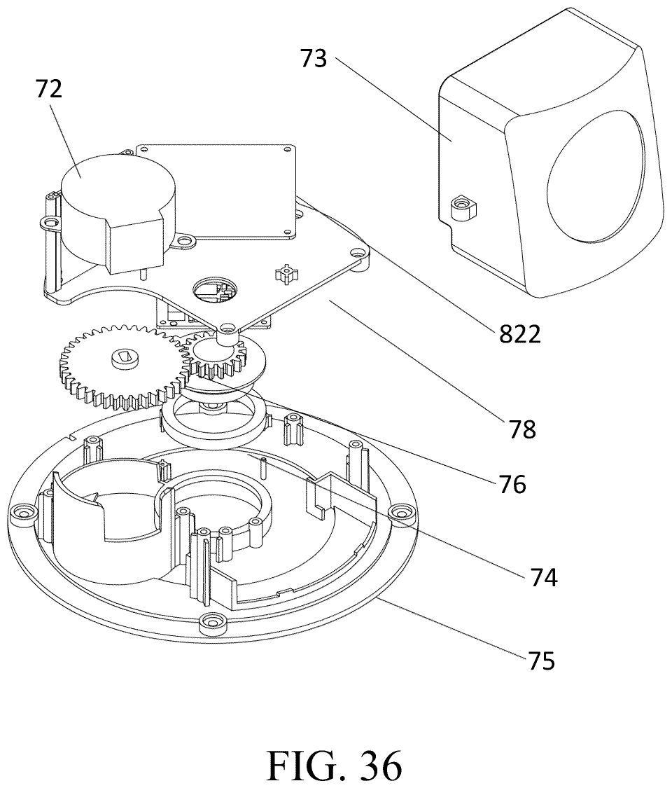

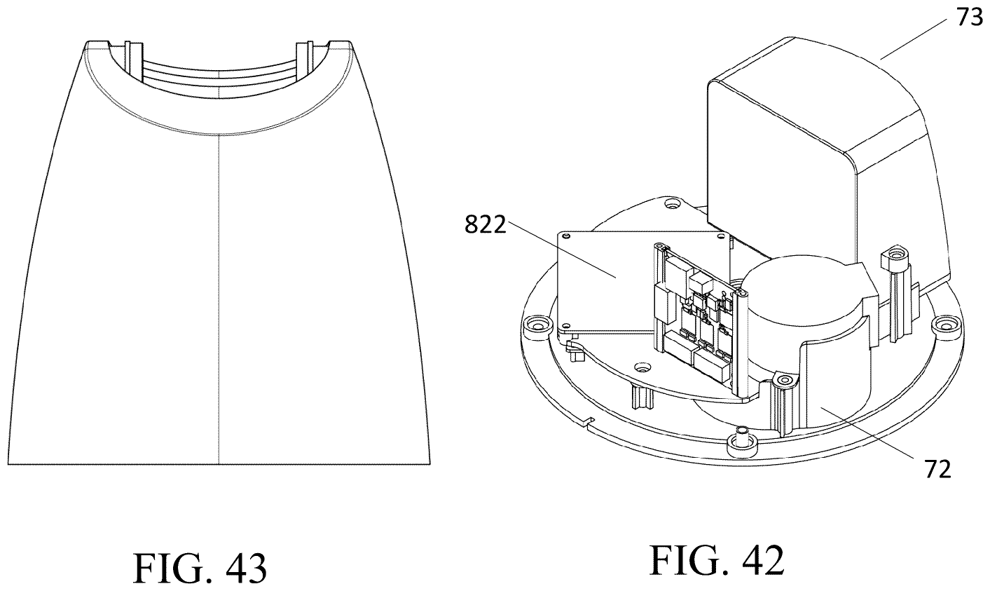

The turntable base 751 is fixed to the base frame 811 by screws; the turntable DC motor and its transmission structure 72, the motor control board 822, and the speaker cavity 73 are fixed to the turntable base 751 by screws, as shown in FIG. 36 and FIG. 42.

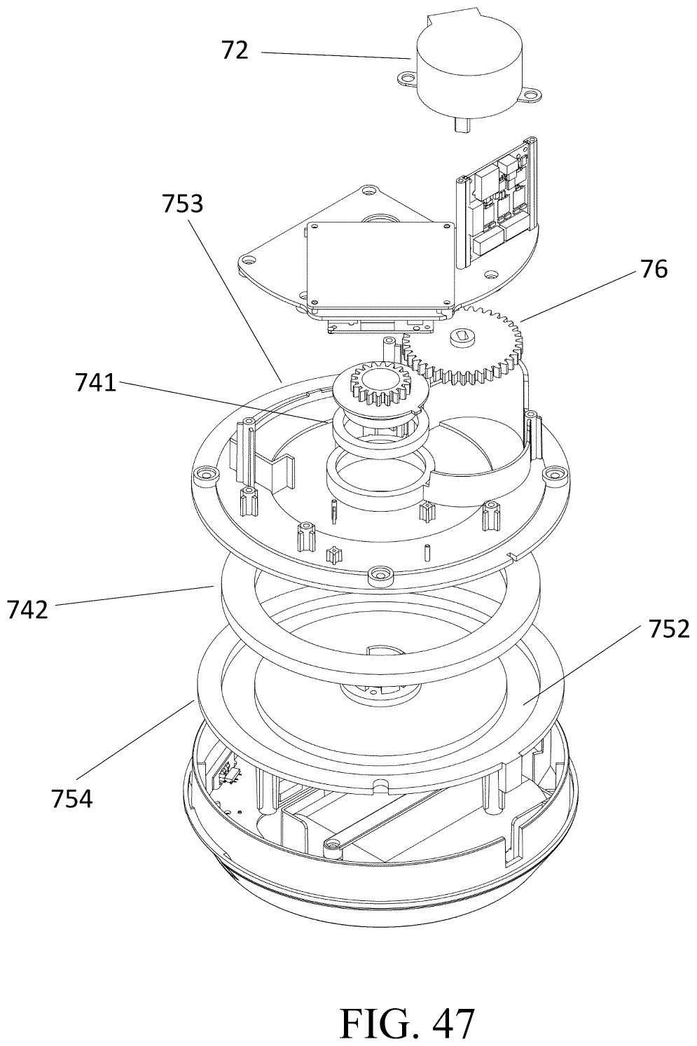

As shown in FIG. 47, the turntable includes an upper turntable 753 and a lower turntable 754. The lower turntable 754 is provided with a groove 752 and is driven by a body gear set 76.

The turntable DC motor and its transmission structure 72 include a turntable DC motor, a gear set, an inner bearing 741, an outer (plane) bearing 742, a turntable motor frame 78, and a motor control board 822;
the inner bearing 741 is connected to the gear and rotates with the gear; the outer bearing 742, also known as the plane bearing, is connected to the body shell to drive the robot body to rotate. The plane bearing also supports the body shell to prevent deformation.

The body shell is connected to the turntable 75 and rotates with the turntable 75. The maximum rotation range is determined by the limit point (the actual maximum rotation angle is 345 degrees).

In the present disclosure, neck shaft fixing frames 77 are arranged on both sides of the inner wall of the body shell, which are used to connect the neck mechanism of the robot, as shown in FIG. 35.

The head, neck and base motors are all connected to the motor control board 822 through wires, and the motor control board 822 is electrically connected to the main control board 31.

Specifically, in the present disclosure,
(1) Battery: Rechargeable battery.
(2) Input power: Type C interface, provides power for the robot.
(3) Output power to charge other devices: two Type C interfaces and one Type A interface.
(4) Machine switch: Turns the machine power on and off.
(5) Power control board: The wires are connected to the battery, the machine switch, and the four USB ports. The power control board is connected to the head main control board through wires.
(6) Network configuration button: Connect to WIFI. Electrically connected to the head main control board through wires.
(7) Turntable base: Place the motor, support the body shell, and rotate with the robot body.
(8) Motor control board: The three motors are electrically connected to the motor control board through wires. The motor control board is electrically connected to the head main control board through wires.

In this embodiment, the relationship between the robot, cloud platform, and application APP

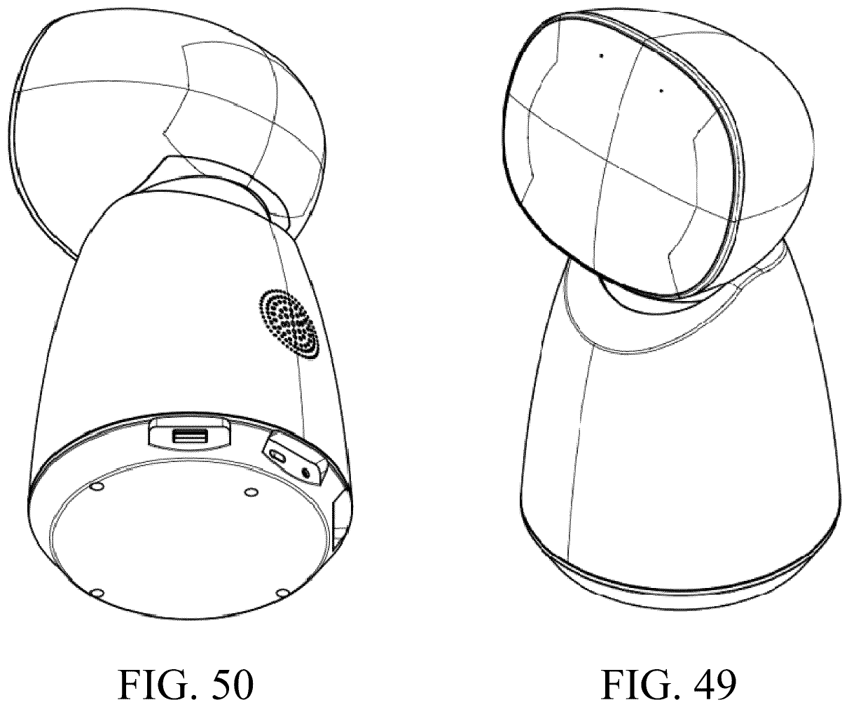

As shown in FIG. 49 and FIG. 50, the intelligent robot of this embodiment is an intelligent terminal with voice input and voice+expression+action output.

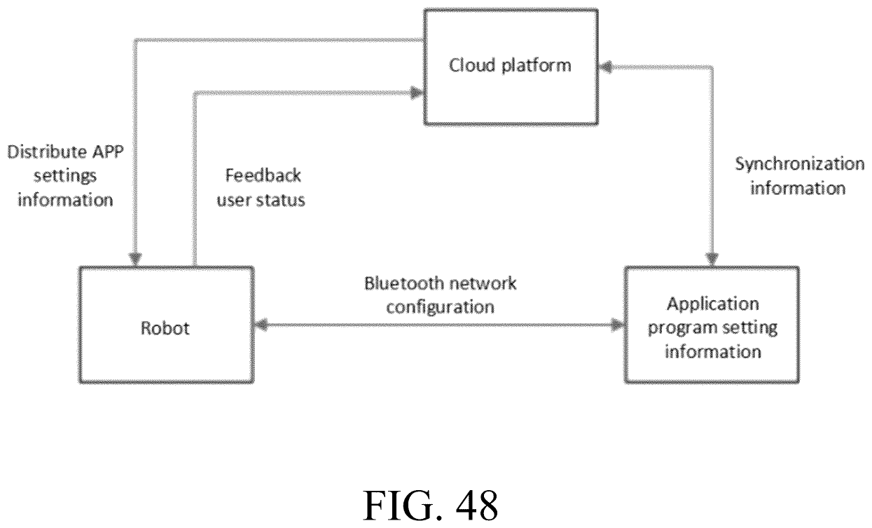

As shown in FIG. 48, the robot, application APP, and cloud platform are interconnected to complete the background data exchange. Information settings are mainly completed on the application APP of the mobile terminal. With the cloud platform as the information transfer center, the application setting information is synchronized with the cloud platform, and then sent to the intelligent robot by the cloud platform. Similarly, the intelligent robot will feedback on the user interaction information to the cloud platform, and then the cloud platform will synchronize it to the application. The intelligent robot needs to access the network through Bluetooth and set up on the application side.

### 2. Medication Reminder Broadcast

The intelligent robot's medication information is set on the application APP, and instructions are sent to the intelligent robot through the cloud platform. When it is time to take the medicine, the intelligent robot will announce the name of the medicine by voice to remind the user to take the medicine on time and in the right amount. The user will give feedback through voice on whether to take the medicine and stop the voice announcement.

The intelligent robot only has the function of timing reminders and has no judgment function to prevent misleading users.

The steps are as follows:
1) The user sets medication information on the APP and synchronizes it to the cloud platform;
2) The cloud platform issues instructions to inform the intelligent robot of the time and medication information to be broadcasted for each medication reminder set by the user;
3) The intelligent robot determines the arrival time of medication based on the pre-stored medication time and system time calibration;
4) The intelligent robot broadcasts medication information, i.e. medication reminders, medicine names, and dosages; at the same time, the intelligent robot locally triggers the screen to display medication reminder expressions, and the robot performs medication reminder supporting actions;
5) Play the medication reminder voice according to the preset number of repetitions and time intervals, turn off the screen after the broadcast, and wait for the user to feedback on medication information;

### Case 1: Taking Medicine on Time, the User Informs the Intelligent Robot by Voice

6) The user has taken the medicine on time, and the intelligent robot is informed by voice
7) Intelligent robots trigger “take medicine on time voice+action+expression”
8) Turn off the screen;
   Case 2: The User does not Tell the Robot Whether they Take the Medicine on Time
9) If the robot does not receive feedback from the user that the medication was taken on time after the preset time, it returns to step 4;
10) After repeating step 4 for the preset number of times, the user still does not receive the instruction from the cloud that the medication has been taken on time.
11) Intelligent robot triggers “not taking medicine on time” voice+action+expression
12) Stop broadcasting and turn off the screen;

### 3. Health Status Query and Report

The daily health monitoring data of the intelligent robot is input into the application APP, and the data is synchronized to the cloud platform. When the user inputs the query demand through voice, the intelligent robot sends the query request to the cloud platform, and the cloud platform sends the health information to the intelligent robot. The intelligent robot broadcasts the health status information through voice.

### 4. Daily Communication and Interaction

The user's voice wakes up the intelligent robot, which turns its body to face the user through sound source and visual positioning, and broadcasts the corresponding voice, expression, and action. The user sends a voice to the intelligent robot, which receives the user's voice information and sends it to the cloud. The cloud interprets the user's voice command, responds differently according to the user's command, and sends the command to the robot to execute the corresponding voice, expression, and action.

The steps are as follows:
1) The user wakes up the intelligent robot through the wake-up word; the wake-up word is recognized locally and the corresponding wake-up operation is performed:
Action+Expression+Voice Expression: Local wake-up expression
Voice: Local built-in voice
Action: The body and head are on one axis, with the head returning to a 90-degree position with the main axis.
2) After the intelligent robot receives the wake-up command locally, it uses sound source and visual positioning to turn its body to face the user, while broadcasting the corresponding voice, expression, and action;
3) The user sends a voice to the intelligent robot;
4) The intelligent robot receives the user's voice and uploads it to the cloud platform;
5) After analyzing the user's voice information, the cloud platform determines whether the user's voice is a query or daily communication;
6) The cloud platform generates answers based on user query information and synthesizes corresponding voice feedback information;
7) The cloud platform sends the answering voice to the intelligent robot, and the command triggers the corresponding expression and action;
8) The intelligent robot plays the voice sent by the cloud platform and triggers corresponding expressions and actions;

### Case 1: User's Voice is a Query Command

9) Turn off the screen after the announcement is completed;

### Case 2: User's Voice is for Daily Communication

9) After the announcement is completed, wait for the user to continue to interact. If there is a new user interaction within the preset time, repeat step 9;
10) After the announcement is completed, if there is no new interaction within the preset time, the intelligent robot will turn off the screen.

### Embodiment 5

The present disclosure provides a desktop-type smart health care system for the field of wise information technology of medicine, including a companion interactive intelligent robot and/or a base and/or an intelligent display processing terminal and/or a multifunctional intelligent medicine box, etc. The intelligent health care system in the present disclosure is capable of collecting, saving and managing daily health indicators, reminding a user in a voice and image manner to measure the health indicators (physical sign health data including, but not limited to, heart rates, blood pressure, blood oxygen, body temperature, and sleep conditions, which are measured by a third-party device) in a timely manner, acquiring third-party device data by means of a communication mode such as Bluetooth and/or WIFI, storing the data in the intelligent display processing terminal, and uploading same to a health care cloud platform; is capable of implementing chronic disease management and, on the basis of daily health data analysis, giving recommendations for chronic disease rehabilitation exercise by using an artificial intelligence algorithm; and is capable of implementing real-time monitoring and early warning of a home environment, including home environment parameters such as gas, water overflow, air quality, etc., and giving a timely voice alarm in case of abnormal conditions. The companion interactive intelligent robot of the smart health care system in the present disclosure implements an interactive companioning function for the user by means of “voices and/or expressions and/or actions”, and can play back information such as user health data, the home environment parameters, and outdoor temperature and weather conditions by means of voices in cooperation with corresponding expressions and actions. The smart health care system in the present disclosure stores and manages medicines in the multifunctional intelligent medicine box, reminds in a voice and image manner the user to take medicine at the appropriate time and in the proper dosage, and sets medication information by means of a WIFI part in a medicine box ESP32 control module. The smart health care system in the present disclosure relates to the technical field of user health care, integrates multiple functions such as health monitoring and management, home environment monitoring and early warning, communication and companion, and is suitable for use in places such as homes and nursing institutions for the user, is well designed for the user, and is convenient for use by the user.

The smart health care system provided in the present disclosure comprises, but is not limited to, the following features:
1) multiple integrated functions are required for daily home health care of the user, including but not limited to, functions such as health monitoring reminder and data collection integration, daily health condition monitoring and analysis management, medication management, home environment monitoring and early warning, voice interaction and companionship;
2) hardware functionality scalability, capable of integrating third-party health monitoring hardware devices (including but not limited to, third-party devices for blood pressure, blood oxygen, blood glucose, sleep monitoring, centimetric wave morphology testing, etc.), providing monitoring parameters for home health care, building a health care device Internet of Things, and expanding system functionality;
3) software functionality scalability, capable of integrating third-party exercise rehabilitation software of chronic disease health care, boosting the medical efficacy of the system, and helping the user achieve chronic disease home health care; and
4) structural properties that can be used in combination or independently, the smart health care system including components such as the companion interactive intelligent robot and/or the base and/or the intelligent display processing terminal and/or the multifunctional intelligent medicine box, the companion interactive intelligent robot being fixed to the base, the intelligent display processing terminal being detachable, the multifunctional intelligent medicine box being allowed to be used separately and independently, the components communicating with one another by means of WIFI, and the health care cloud platform being used as a data transfer platform, thus enhancing the flexibility for use in the home environment.

By using independently designed hardware applied to the usage scenarios of smart health care, and by virtue of technologies such as the Internet of Things and artificial intelligence, the smart health care system for the field of wise information technology of medicine in the present disclosure provides an integrated implementation solution in the field of smart health care including health monitoring and management, medication management, interactive companionship, environmental monitoring and early warning, etc.

### Embodiment 6

In one specific embodiment of the present disclosure, the intelligent medicine box end communicates with the health care cloud platform to acquire medication information of the user, reminds the user of medicine types, dosages, medication precautions, etc. at set times and, after detecting that the user has taken medicine from the intelligent medicine box, synchronizes information to the health care cloud platform, avoiding over- or under-medication of the user due to memory deterioration.

The care robot end communicates with the health care cloud platform, triggers interactions with the user in a specified scenario, executes specified action and expression instructions according to the interaction scenarios, and conducts conversations with the user in an anthropomorphically vivid way in light of the scenarios, so as to remind the user of daily matters, assist in looking for information or acquiring services, and providing daily care for the user in a more warm and lively manner.

The home intelligent end communicates with the intelligent medicine box end and/or the care robot end and/or the access device, communicates with the family end and/or the service back end via a cloud platform, implements functions including, but not limited to, health management and/or medication management and/or exercise rehabilitation and/or sleep management and/or video calling and/or health reporting and/or emergency calling and device linking, etc., which are implemented by modules including, but not limited to, a health management module and/or a medication management module and/or an exercise rehabilitation module and/or a sleep management module and/or a video calling module and/or a health report module and/or an emergency calling module and/or a device linking module, etc. In actual implementation, certain modules may be integrated so as to implement the functions by using one module capable of implementing multiple functions.

The health management module collects the health data of the user and uploads a data record to the health care cloud platform, providing real and valid health data for a health report generated by the health care cloud platform; and faithfully records examination results, aiding the user in knowing about their own health conditions and providing doctors with comprehensive and detailed medical historical data, which helps to make further diagnosis and effective treatment;
after collecting a medicine prescription for the user, the medication management module generates a corresponding medication schedule according to the prescription, and will remind, after synchronizing the medication schedule to the health care cloud platform, at the scheduled time the user to take medicine in cooperation with the intelligent medicine box according to the medication schedule and, after the user take medicine, synchronizes a result of medication, helping family members and the user understand daily medication situations and medication history;
the exercise rehabilitation module provides individual targeted professional exercise prescriptions for the user in need of exercise rehabilitation, reminds the user to take exercise according to exercise prescription content, and records exercise data and physical sign data of the user and uploads same to the health care cloud platform, so as to aid rehabilitation doctors in effectively adjusting the exercise prescriptions in a timely manner according to exercise situations to achieve the purpose of exercise rehabilitation;
the sleep management module collects real-time physiological indicators and in/out-of-bed states during the sleep process of the user; once discovering heart rate and respiration abnormalities or in/out-of-bed time exceeding a threshold at night, synchronizes data to the health care cloud platform in real time to give an alarm, so as to avoid an accident during the sleep process at night and ensure the safety of the user at night; and may analyze sleep data to provide the user suffering parahypnosis with a medical intervention program for sleep improvement;
the video calling module may initiate/answer a video call to/from the family end at any time by means of a smart terminal, and may also make a one-touch call to acquire various services from a service platform, so as to meet daily communication and user-friendly service needs of the user and improve the quality of life of the user;
the health report module receives cloud platform report information, and presents data including a physical sign report, a medication report, an exercise report, and a sleep report to the user in a voice playback and page display manner, helping the user know about their own health conditions truly and comprehensively, avoiding unnecessary tension, fear or blind optimism of the user due to a lack of understanding of their own health conditions, and reminding the user to pay attention to reasonable diets, to take proper exercises and to keep a good state of mind;
after detecting an abnormal condition, the emergency calling module automatically triggers an alarm and synchronizes information to a platform operation management and control module and the family end, and the user may also initiate an emergency call by voice or by a one-touch button, aiding family members or related institutions in discovering abnormality in time for making valuable rescue time;
and the device linking module is connected to a specified medical device by means of a Bluetooth/WIFI protocol, acquires relevant physical sign data, and may help the user to record relevant physical sign data information more conveniently and quickly.

The health care cloud platform provided by the present disclosure is a user-oriented smart health care system platform based on the Internet of Things and cloud computing, providing functions including, but not limited to, health management and/or medication management and/or alarm management and/or report management and/or algorithm management and/or device linking management and/or user management and/or family management and/or operation management and/or data management and/or video calling management, etc.

The health management is used for collecting health data of the user for data collection and recording, and summarizing the data for generating a health report, the health data including, but not limited to, physiological indicators and/or a data acquisition manner and/or acquisition time, etc.;
the medication management is used for the user to acquire medication schedules and actual medication situations of the user, to record medication data in a cell phone, and to summarize the medication data for generating a medication report, medication information including, but not limited to, the medication name and/or a medication frequency and/or the medication time and/or the dosage and/or a medication contraindication and/or a medication result, etc.;
the alarm management is used for receiving alarm information of a 99 health care intelligent end in real time and, by means of making an alarm call or pushing the alarm information, notifying the family end or an operation management end of the alarm information according to types;
the report management is used for generating health reports of the user. The health reports are generated according to different statistical periods and include daily reports, weekly reports, monthly reports, quarterly reports, semiannual reports and annual reports; and report contents include, but are not limited to, physiological indicator parameters and variation conditions and/or medication information record and medication situation statistics and/or exercise rehabilitation program and execution results and/or sleep information statistics and abnormal condition summary and sleep improvement advice, etc.;
the algorithm management is used for model management and training of platform AI algorithms, including but not limited to a voice recognition algorithm and/or a situational dialogue algorithm and/or a health information recommendation algorithm, etc.;
the device linking management is used for management, including but not limited to, device name and/or type and/or communication mode and/or protocol management, etc., of an access device that communicates with a home intelligent end device, and is also used for control of the access device linked by the user;
the user management is used for querying data of the user, the information of the user, including but not limited to, basic user information and/or health information and/or family information and/or device linking information, etc., and a query mode being as follows: acquiring user information of the user, displaying a role after matching the information in a data query system, extracting and displaying a service template carried by the user role in the data query system, and displaying corresponding user query content in the corresponding service template, thus obtaining a query result;
the family management is used for querying the linking relationship between the family user and the user in the following mode: acquiring family end user information and, after matching the information in the query system, displaying information of the linked user;
the operation management is used for back-end operation platform management and access to a third-party service resource, including but not limited to, access to a call center, and/or access to a service agency or platform, and/or access to a life service;
the data management is used for summarizing data uploaded by a cooperating smart device and a linked device and, after classified summarization of the data, storing the data in the cloud and pushing the data processed by an algorithm to a corresponding service module for use;
and the video calling management is used for processing a video call initiated by the home intelligent end or the family end: after an initiator initiates a call, the health care cloud platform identifies a called user according to the user linking relationship and gives a ring prompt of a conversation request to a corresponding called user end and, after the called user accepts the call, establishes a video call link and records information, including but not limited to, call initiation time and/or an initiating user and/or the called user and/or call duration, etc.

The family end receives, by means of the smart terminal, the health reports authorized by the home intelligent end, so as to know about the physical condition, medication situation and exercise rehabilitation condition of the user in a timely manner and to know about the basic body condition of the user, avoiding blindly optimistic estimation or unnecessary fear or tension, allowing a clear understanding and a correct attitude; and may make contact with the user at home anytime by means of the video calling function, helping the family members to know about the conditions of the user anytime and anywhere, helping to communicate with the user at home, providing warmness and care, and satisfying mental needs of the user.

### Embodiment 7

A process of a mechanical system's nodding action is provided. The complete description is as follows:
1. Trigger nodding action: The process starts with a certain trigger condition, initiating the nodding action.
2. Check if the head, neck, and body are in the starting position: The system first checks whether the head, neck, and body are in the starting position. If not, the next operation is executed.
3. The motor drives the head, neck, and body back to the starting position: If the head, neck, and body are not in the starting position, the motor drives them back to the initial position.
4. The motor drives the neck motor: After confirming that the head, neck, and body have returned to the starting position, the motor begins to drive the neck motor to prepare for the nodding action.
5. Adjust the motor's rotational speed and amplitude: After the neck motor is driven, the system adjusts the motor's rotational speed and amplitude to complete the nodding action.
6. The neck motor drives the motor gear: The neck motor further drives the motor gear through the gear mechanism, causing it to rotate.
7. The motor gear drives the transmission gear: The motor gear's rotation drives the transmission gear to continue turning, and transmitting power.
8. The transmission gear drives the bearing: The transmission gear drives the bearing, which is fixed to the outer shell of the neck, causing movement.
9. The neck and head are fixedly connected, and the neck drives the head to move: the neck and head are fixedly connected, so the neck's movement drives the head to move, completing the nodding action.
10. The head and neck return to the starting position: After the nodding action is completed, the head and neck are driven back to the starting position, preparing for the next nodding action.
11. Complete one nodding action: The system records the completion of this nodding action.
12. Nodding action count+1: The system records the number of nodding actions performed, incrementing the counter by one.
13. Check if the predetermined number of repetitions is reached: The system checks whether the number of nodding actions has reached the preset threshold. If it has, the process moves to the next step; if not, the system returns to repeat the nodding action.
14. Complete the nodding action: Once the nodding action has reached the predetermined number of repetitions, the entire nodding action process ends.

### Embodiment 8

An operation process of a robot head touch-trigger system, describing several response steps after the robot receives a touch command, is provided. The detailed description is as follows:
1. Touch the robot's head: The process is initiated when a user or external trigger touches the robot's head.
2. Main control board: The touch signal is transmitted to the main control board, which processes the signal and coordinates the subsequent actions.
3. Display shows cute expression: Based on the touch signal, the main control board controls the display to show a cute expression as visual feedback.
4. Speaker plays cute background sound: Simultaneously, the main control board also controls the speaker to play background sound that matches the expression, enhancing the interactive experience.
5. The motor driver board triggers corresponding actions: The main control board directs the motor driver board to initiate mechanical actions related to the touch signal:
1) Head motor shakes the head: After touching the head, the robot's head motor performs a shaking action.
2) Neck motor nods the head: The neck motor executes a nodding action as part of the mechanical feedback.
3) Base motor rotates the body: The base motor drives the entire robot's body to rotate, coordinating with the head and neck movements.

Although the present invention has been described in considerable detail with reference to certain preferred embodiments thereof, the present disclosure is not to limit the scope of the present invention. Persons having ordinary skill in the art may make various modifications and changes without departing from the scope and spirit of the present disclosure. Therefore, the scope of the appended claims should not be limited to the description of the preferred embodiments described above.

What is claimed is:

1. A companion interactive intelligent health care system, comprising: a hardware part and a software part;
the hardware part comprising: a multifunctional intelligent medicine box, a companion interactive intelligent robot, and an intelligent display processing terminal; wherein,
the medicine box is used to display a name and dosage information of a medicine, indicate a medicine compartment corresponding to the medicine, and record medication information;
the companion interactive intelligent robot integrates voice, expression, and action, and is used to remind a user to take the medicine on time and in a right amount through multiple sensory interactions, provide emotional companionship, and remote communication with family members of the user; and
the intelligent display processing terminal is used for system software operation, data acquisition, storage, and analysis, remote viewing and monitoring of medication records of the user, or any combination thereof; and
the software part integrating Internet of Things technology and artificial intelligence technology, and comprising a health care cloud platform, a family end, a service back end, a home intelligent end, an intelligent medicine box end, a robot end, or any combination thereof.

2. The companion interactive intelligent health care system of claim 1, wherein the medicine box comprising: a machine head and at least one medicine storage box;
the machine head comprises: a machine head body and a machine head base;
the medicine storage box comprises: a box shell and a medicine storage drawer, wherein the medicine storage drawer can be pulled out and moved inside the box shell;
the machine head matches a top structure of the medicine storage box, so that the machine head can be fixedly connected to the medicine storage box;
the medicine storage drawer comprises: a medicine storage drawer body; the medicine storage drawer body comprises: a front panel and a drawer body; the front panel is installed at the front end of the drawer body;
the front panel is provided with a drawer handle, and the side surface of the drawer body is provided with a sliding groove; the sliding groove corresponds to a box slide rail provided inside the box shell;
a medicine box status LED light is provided at the top of the front panel, and the medicine box status LED light can display different colors according to different scenes; when there are multiple layers of medicine boxes, the medicine box status LED light can be turned on to indicate the corresponding medicine box storing the medicine.

3. The companion interactive intelligent health care system of claim 2, wherein, inside the medicine storage drawer body is divided into medicine compartments of different sizes by pluggable partition boards; a protrusion is provided on the upper part of the partition board of each medicine compartment, and a partition board LED button light is provided at the protrusion; after the medicine is put in or taken out, the partition board LED button light can be pressed to indicate the position of the medicine; a partition board LED button light backboard is provided at the back of the partition board corresponding to the partition board LED button light for controlling the partition board LED button light.

4. The companion interactive intelligent health care system of claim 2, wherein the machine head base is a T-shaped structure, which matches a U-shaped slide rail structure protruding from the top of the medicine storage box, and the machine head base is inserted into the U-shaped slide rail structure at the top of the medicine storage box through a positioning buckle at the bottom of the machine head base and a positioning slot at the top of the medicine storage box; and
the front of the head body is provided with a display screen, a scanning camera, function buttons, and a volume adjustment button.

5. The companion interactive intelligent health care system of claim 4, wherein a display screen storage box is provided at the upper end of the machine head base, and the display screen matches the size of the display screen storage box; the display screen is movably connected to the machine head base through a hinge and a damping bracket, and can be stored in the display screen storage box; the damping bracket is installed on the back of the display screen to control a lifting angle of the display screen, and the lifting angle ranges from 0 to 90°.

6. The companion interactive intelligent health care system of claim 4, wherein two sides of the T-shaped structure of the machine head base can be slid and snapped into an inner side of the slide rail of the U-shaped slide rail structure at the top of the medicine storage box; a tail of the machine head base is a step-shaped structure, which matches and snaps with a bottom shape of the U-shaped slide rail structure at the top of the medicine storage box; the positioning buckle at the bottom of the machine head base corresponds to the positioning slot at the top of the medicine storage box, and when the machine head is fixedly connected to the medicine storage box, the positioning buckle snaps into the positioning slot;
the step-shaped structure of the machine head base is provided with a pin interface which contacts a power supply pin at the bottom of the U-shaped slide rail structure at the top of the medicine storage box to supply power to the medicine storage box; the pin interface of the machine head is connected to a power control board via a wire; and
a control mainboard and the power control board are fixedly arranged at the back of the display screen inside the machine head body; the control mainboard comprises a voice module, a display module, and a WIFI module;
a lithium battery, a Bluetooth antenna array board, and a speaker cavity are fixedly arranged at the bottom of the interior of the machine head body; the speaker cavity is installed above the Bluetooth antenna array board and corresponds to a speaker hole on the back of the machine head body;
the control mainboard, the power control board, the lithium battery, the Bluetooth antenna array board, and the speaker cavity are electrically connected, and processed signals transmitted therebetween are aggregated to the control mainboard;
the voice module is used to recognize and generate sounds; the display module is used to display status information of the medicine box, medication-related information, and input medication information; the WIFI module is used to transmit device information and medication management information to the cloud platform; the power control board is used as an electrical connection transfer structure to monitor battery power, power connection status, and machine switch status;
the Bluetooth antenna array board is used to transmit Bluetooth communication signals to achieve Bluetooth communication between the machine head and the medicine storage box.

7. The companion interactive intelligent health care system of claim 6, wherein the U-shaped slide rail structure and the positioning slot are arranged on the top of the box shell of the medicine storage box, and a power supply pin is arranged at the bottom of the U-shaped slide rail structure on the top of the medicine storage box; a T-shaped structure base is arranged at the bottom of the box shell of the medicine storage box, and a box installation buckle is arranged at the bottom of the T-shaped structure base, and a box bottom pin is arranged at the rear of the T-shaped structure base; the T-shaped structure base corresponds to the U-shaped slide rail structure, and the box bottom pin contacts with the power supply pin at the bottom of the U-shaped slide rail structure on the top of other medicine storage boxes to supply power to the other medicine storage boxes; the pin interface of the machine head is connected to the power control board through the wire;
a rear portion of the box shell is provided with another box bottom pin, a power distribution board, and a Hall sensor magnet;
the box bottom pin is arranged at a lower part of the rear portion of the box shell; the power distribution board includes three circuit interfaces, one of which inputs a power supply signal from the power supply pin at the top of the medicine storage box, and the other two are respectively connected to the pins at the bottom of the box through wires, and connected to a Bluetooth main control board on the medicine storage drawer through a spring power line, respectively supplying power to the medicine storage box and the Bluetooth main control board; and the Hall sensor magnet corresponds to the Hall sensor board arranged on the medicine storage drawer.

8. The companion interactive intelligent health care system of claim 2, wherein the medicine storage drawer further comprises: a Hall sensor board and a Bluetooth main control board; the Hall sensor board is installed at the rear of the medicine storage drawer body, and the Bluetooth main control board is installed at the bottom of the medicine storage drawer body;
the Hall sensor board is electrically connected to the Bluetooth main control board at the bottom of the medicine storage drawer; the Hall sensor board corresponds to a Hall sensor magnet provided on the box shell, and senses opening and closing states of the medicine storage drawer through contact between the Hall sensor board and the Hall sensor magnet, and transmits state signals to the Bluetooth main control board through a wire;
the Bluetooth main control board receives the status signals of a partition board LED button light and opening and closing status signals of the medicine box by the Hall sensor, and sends the received signals to a Bluetooth antenna array board or a Bluetooth module of the machine head, and then the Bluetooth antenna array board or the Bluetooth module sends the signals to a control main board of the machine head; the Bluetooth main control board supplies power to the partition board LED button light and the Hall sensor board.

9. The companion interactive intelligent health care system of claim 2, wherein a medicine box attitude testing limit switch is provided inside the medicine box, and the medicine box attitude testing limit switch can be used to count the number of times of opening and closing of the medicine box.

10. The companion interactive intelligent health care system of claim 1, wherein the companion interactive intelligent robot comprises: a head mechanism, a neck mechanism, and a body mechanism; the head mechanism and the body mechanism are connected via the neck mechanism;
the head mechanism comprises: a display screen, a microphone, a camera, and a capacitive touch sensor, which are used for user interaction and voice or visual feedback, and rotation and shaking of the head mechanism are controlled by a motor;
the neck mechanism controls movements of the head mechanism in different directions, and comprises: a neck DC motor, a neck gear set, and a neck bearing structure; and
the body mechanism supports the overall rotational movement of the robot, and comprises: a turntable DC motor and transmission structure thereof, and a turntable.

11. The companion interactive intelligent health care system of claim 1, wherein the intelligent display processing terminal is detachable and can be carried and used alone;
the intelligent display processing terminal is capable of running system software, acquiring, saving, and analyzing daily health monitoring data, communicating data with the cloud platform, and communicating with system hardware devices;
the intelligent display processing terminal and the companion interactive intelligent robot realize scenario-triggered interactive operations through WIFI, and the intelligent display processing terminal sends to the robot voice broadcast contents, comprising user health measurement data and home environment monitoring data;
data transmission between the intelligent display processing terminal and the medicine box is transferred via WIFI using the cloud platform; and
the intelligent display processing terminal and a third-party health monitoring hardware equipment realize data collection through WIFI/Bluetooth communication technology.

12. The companion interactive intelligent health care system of claim 1, further comprising a base, wherein the base is provided with a companion interactive intelligent robot interface, an intelligent display processing terminal interface, or a combination thereof; the companion interactive intelligent robot can be rotatably fixed on the base through bolts, and the intelligent display processing terminal is fixed to the base via magnetic attraction or charged via a base charging socket provided on the base;
the base is provided with a fitting base support with adjustable angles and directions, and the intelligent display processing terminal can be fixed to the base via the fitting base support;
a back surface of the base is provided with a base power switch, the base power switch is used for controlling on-and-off of the base and of the robot;
the base is provided with a base groove, the intelligent display processing terminal is in contact with the base via the base groove.

13. The companion interactive intelligent health care system of claim 12, wherein the base further comprises a robot control module, an Android development board, a base loudspeaker, or any combination thereof;
wherein the robot control module comprises a base ESP32 control module, a voice recognition module, a head liquid crystal screen control module, a motor driving chip, or any combination thereof;
the base ESP32 control module is used for implementing WIFI and Bluetooth functions;
the voice recognition module is used for recognizing voice so as to implement an interaction with the robot, and playing a synthesized voice;
the motor driving chip is used for driving a motor inside the robot to move;
the Android development board is used for providing a control function for the voice, the screen, the motor, or any combination thereof, and implementing WIFI and Bluetooth functions; and
the base loudspeaker is used for playing the synthesized voice.

14. The companion interactive intelligent health care system of claim 1, wherein the health care cloud platform is an integrated system platform capable of bi-directionally communicating with the family end, the service back end, the home intelligent end, or any combination thereof; the home intelligent end is in bi-directional communication with the intelligent medicine box end and the robot end;
the health care cloud platform is used for providing functions comprising health management, medication management, alarm management, report management, algorithm management, device linking management, user management, family management, operation monitoring, data management and control, message management, video calling, or any combination thereof;
the family end is mounted on a smart device and is used for implementing functions comprising linking with the user, receiving health reports of the user, video calling with the user, or any combination thereof;
the service back end is used for management of a system back end and access to a third-party service resource, and is capable of providing functions comprising call center calling, service allocation, service access, or any combination thereof;
the home intelligent end comprises a health management module, a medication management module, an exercise rehabilitation module, a sleep management module, a video calling module, a health report module, an emergency calling module, a device linking module, or any combination thereof, and is used for implementing functions comprising health management, medication management, exercise rehabilitation, sleep management, video calling, health reporting, emergency calling, device linking, or any combination thereof;
the intelligent medicine box end communicates with the health care cloud platform, and is capable of acquiring medication information of the user, reminding the user of medicine types, dosages, medication precautions, or any combination thereof, at set times and, after detecting that the user has taken medicine from the intelligent medicine box, synchronizing information to the health care cloud platform, so as to implement functions including medication reminding and medication statistics; and
the robot end communicates with the health care cloud platform, and is capable of triggering interactions with the user under a preset environment, and executing a corresponding action and expression instruction according to an interactive scenario, so as to implement functions comprising voice recognition, situational dialogue, care reminding, action and expression presentation, or any combination thereof.

15. The companion interactive intelligent health care system of claim 14, wherein the smart health care system is further capable of importing data in an external access device via a device access protocol.

16. An intelligent medication management method of the companion interactive intelligent health care system of claim 1, comprising steps of:
setting medication information in the intelligent display processing terminal, sending instructions to the companion interactive intelligent robot through the cloud platform, and when reaching a medication time, playing by the companion interactive intelligent robot a voice reminder to the user to take the medicine, receiving voice feedback from the user on taking the medicine or not, and stop playing the voice reminder;
when a preset medication time has come, playing the name and dosage information of the medicine by the medicine box, lighting up the screen to display the name and dosage information of the medicine, and turning on a medicine box status LED light at the front of a medicine storage drawer of a medicine storage box of the medicine box to remind the user to open the drawer and take the medicine; and
after the medicine storage drawer is opened, turning on a partition board LED button light of a medicine compartment, and after the medicine is taken and put back, closing the medicine storage drawer when the partition board LED button light is pressed, turning off the partition board LED button light and the medicine box status LED light to indicate that the medicine has been taken on time, sending a signal to the cloud platform to record the medication situation, and generating a medication report.

17. The method of claim 16, wherein the medication report of the user is remotely viewed through an intelligent display processing terminal;
during medication, a function button of a machine head below a display screen of the medicine box can be pressed to stop the voice playing by the companion interactive intelligent robot;
when the medicine is not taken at the preset medication time, the medicine box is configured to play multiple voice reminders and issuing an alarm;
a camera on the medicine box is configured to scan medicine barcodes to query the medication information and check whether a medication plan has been set; if the plan has been set, the medication information and medication arrangement would be prompted through voice and screen display; if no plan has been set, the user would be asked whether to set up the plan and be guided to enter information including medicine name, dosage, and time, and the information would be synchronized to the cloud platform to ensure remote monitoring.

18. The method of claim 16, wherein the companion interactive intelligent robot comprises companionship interaction, daily life information query and broadcast, user health information query and broadcast, medication information reminder query and broadcast, and remote interactive call functions;
the companion interactive intelligent robot interacts with the user through scenes, voices, expressions, and actions, and performs actions under specific scenes;
the companion interactive intelligent robot is connected to the cloud platform and an application (APP), the user sets a medication reminder through the APP, and the cloud platform issues instructions, the companion interactive intelligent robot broadcasts medication information on time, if the user does not give feedback, the companion interactive intelligent robot would repeatedly send out reminders; and
the user wakes up the companion interactive intelligent robot by voice to inquire about daily health conditions or seek interaction, and the cloud platform analyzes user's instructions and generates feedback.
